SEQUENCE LISTING (18 sequences)

SEQ ID NO: 1 - protein, 184 aa, Triticum aestivum
MEREATVAFYPPAPAPQQQRPTAAQPLSARAPSSGGGVPAGRGGGGRQYRGVRMRKWGKWVAEIREPNKRSRIWLGSYATAVAAARAYDTAVFYLRGRSARLNFPDQLLDGAPAAAAPGDLTAAAIRKKAAEVGARVDALHSGGIGVGIGMGGAPPAPPSPSQRRRAKNPNLNREPTSDTDDDQ

SEQ ID NO: 2 - DNA, 555 bp, Triticum aestivum
atggagagggaagccacggtcgccttctacccgccggcgccggcgccgcagcagcagcgccccacggccgcgcagccgctctcggcccgcgcgccttcgtccggcggcggcgtccccgcggggcggggaggcgggggcaggcagtaccgcggggtgcggatgcgcaagtggggcaagtgggtggccgagatccgggagcccaacaagcggtcgcggatctggctcggctcctacgccaccgccgtcgccgccgcgcgcgcctacgacaccgccgtcttctacctccgcggccgctccgcgcgcctcaacttccccgaccagctcctcgacggcgcccccgccgccgccgcgcccggggacctcaccgccgccgccatccgcaagaaggccgccgaggtcggcgcgcgcgtcgacgcgctccactccggcgggatcggcgtcggcatcggcatgggcggcgcccccccggccccgccgtcgccgtcccagcgccgccgggccaagaaccccaacctcaaccgggagcccacctccgacaccgacgacgaccagtga

SEQ ID NO: 3 - protein, 179 aa, Triticum aestivum
MEREATVAFYPPAPAPQQQRPTAAQPLSARVPAGGVHAGRGGGGRQYRGVRMRKWGKWVAEIREPNKRSRIWLGSYATAVAAARAYDTAVFYLRGRSARLNFPDQLLDGTAPAAAPGDLTAAAIRKKAAEVGARVDALHSGGIGVGMGAPAAPPSPSQRRRAKNPDLNREPTPDTDDDE

SEQ ID NO: 4 - DNA, 540 bp, Triticum aestivum
atggagagggaagccacggtcgccttctacccgccggcgccggcgccgcagcagcagcgccccacggccgcgcagccgctctcggcccgcgtgcctgccggcggcgtccacgcgggccggggaggagggggcaggcagtaccgcggggtgcggatgcgcaagtggggcaagtgggtggccgagatccgggagcccaacaagcggtcgcggatctggctcggctcctacgccaccgccgtcgccgccgcgcgcgcctacgacaccgccgtcttctacctccgcggccgctccgcgcgcctcaacttccccgaccagctcctcgacggcactgcccccgccgccgcgcccggggacctcaccgccgccgccatccgcaagaaggccgccgaggtcggcgcgcgcgtcgacgcgctccactccggcggcatcggggtcggcatgggcgcccctgcggcaccgccgtcgccgtcccagcgccgcagggccaagaaccccgacctcaaccgggagcccacccccgacaccgacgacgacgagtga

SEQ ID NO: 5 - protein, 243 aa, Triticum aestivum
MQQGEYRSSSSSEGSAGSAAAAAAAAAMAPLAAAAAAVAAKEEHNVTVAVAPPMPMAMAMPLQQQQPRKQYRGVRMRKWGKWVAEIREPHKRTRIWLGSYATPVAAARAYDTAVFYLRGRSARLNFPDEISALALSSPEAAEAGGGEEPGGGGGALSAASIRKKAIEVGSRVDALQTGMTTMVAAPAHHRERQRLHHHHHHAEPHGEELHRHVKQQRTAWNGRAKNPDLNQAPSPDTSDAEAE

SEQ ID NO: 6 - DNA, 732 bp, Triticum aestivum
atgcagcagggcgagtaccgctcgtcgtcttccagcgagggctccgcggggtcggctgctgcggctgcggcggccgcggccatggcgcccctggcggctgcggccgcggcggtggcggccaaggaggagcacaacgtgacggtggccgtggcgccgcccatgcccatggcgatggcgatgccgctgcagcagcagcagccgcggaagcagtaccgcggcgtgcgcatgcgcaagtggggcaagtgggtggcggagatccgcgagccgcacaagcgcacgcgcatctggctgggctcctacgccacccccgtcgccgccgcgcgcgcctacgacacggccgtcttctacctgcgcggccggtcggcgcgcctcaacttccccgacgagatctccgcgctggcgctgtcgtcgcccgaggccgccgaggccggcggaggggaggagccgggcggcggcggcggcgcgctgtcggccgcgtcgatccggaagaaggccatcgaggtcgggtcccgcgtggacgcgctgcagaccggcatgaccaccatggtcgccgcgcccgcgcaccaccgcgagcggcagcggctccaccaccaccaccaccacgcggagccgcacggcgaggagctgcaccgccacgtgaagcagcagcggacggcgtggaacgggcgcgccaagaacccggatctcaaccaggcgccgagcccggacacctccgacgccgaggccgagtga

SEQ ID NO: 7 - protein, 249 aa, Zea mays
MQGEYRSSSEESAAAAAAAAMAPLAAAAAAAVKMEAEQAAMASAAAPQLGSAHQHQHQQMQPRRQYRGVRMRKWGKWVAEIREPHKRTRIWLGSYATAVAAARAYDTAVFYLRGRSARLNFPDEIPSLAPSEGEGEGDGGEPARDPADGGGTLSAASIRKKAIEVGSRVDALQTGMMVPPPHHRERQRHHHHHHHHLPQLRMHAAAAEAAAVAEHRQEVKQSPQRPAWSGRVKNPDLNRAPSPESSDAE

SEQ ID NO: 8 - DNA, 750 bp, Zea mays
atgcaaggcgagtaccggtcttccagcgaggagtcggcggccgctgcagcagccgcagccatggctcctctggcggccgccgcggccgcagcagtgaagatggaggcggagcaggcggcgatggcgagcgcggctgcgccgcagctggggtcggcgcaccagcaccagcaccagcagatgcagccgcggcggcagtaccgcggcgtgcgcatgcgcaagtggggcaagtgggtggcggagatccgggagccgcacaagcgcacgcgcatctggctggggtcctacgccacggccgtggcggcggcgcgcgcctacgacacggccgtgttctacctgcgcggccggtcggcgcggctcaacttccccgacgagatcccgtcgctggcgccgtcggagggcgagggcgagggcgacggcggggagcccgcgcgcgaccccgccgacggcggcggcacgctgtccgccgcgtccatccggaagaaggccatcgaggtggggtcccgcgtggacgcgctgcagaccggcatgatggtgccgccgccgcaccaccgcgagcgccagaggcaccaccaccaccaccaccaccacctgccgcagctgcggatgcacgccgccgccgcggaggcggcggctgtggcggagcaccggcaggaggtgaagcagtcgccgcagcggccggcgtggagcgggcgcgtcaagaacccggatctaaatcgggcgcccagccccgagagctccgacgccgagtga

SEQ ID NO: 9 - protein, 227 aa, Zea mays
MQGEYRSSSEESAAAAAVAAAAAMAPLAAAEVKMEAEQVAMAAPQLGAAHQQTQPRRQYRGVRMRKWGKWVAEIREPHKRTRIWLGSYATAVAAARAYDTAVFYLRGRSARLNFPEEIPSLGLADGVDVGEPARSGGGGGCTLSAASIRKKAIEVGSRVDALQTGMVVPPPHHRERHRHHNHLPQLRGHAHAHAEEQQQQPQRPVWSGRVKNPDLNRAPSPESSDAE

SEQ ID NO: 10 - DNA, 684 bp, Zea mays
atgcagggcgagtaccggtcttccagcgaggagtcggcggcggccgccgctgtagccgcagccgcagccatggctcctctggcggccgcggaggtgaagatggaggcggagcaggtggcgatggcggcgccgcagctgggggccgcgcaccagcagacgcagccgcggcggcagtaccgcggcgtgcgcatgcgcaagtggggcaagtgggtggcggagatccgggagccgcacaagcgcacgcgcatctggctggggtcctacgccacggccgtggcggcggcgcgcgcctacgacacggccgtgttctacctgcgcgggcggtcggcgcggctcaacttccccgaggagatcccctcgttagggctggcggatggcgtggacgtgggggagcccgcgcgctccggcggcggcggcggatgcacgctgtccgcggcgtccatccggaagaaggccatcgaggtggggtcccgcgtggacgcgctccagaccggcatggtggtcccgccgccgcaccaccgcgagcgccataggcaccacaaccacctgccgcagctgcgggggcacgcgcacgcgcacgcggaggagcagcagcagcagccgcagcggcctgtgtggagcgggcgcgtcaagaacccggatctgaaccgtgcgccgagccccgagagctccgacgccgagtga

SEQ ID NO: 11 - protein, 233 aa, Zea mays
MMQGEYRSSIEESAAAAVAAAAAMAPLAAAAVKMEAEQAAMAAPQLGAAHQQTQPRRQYRGVRMRKWGKWVAEIREPHKRTRIWLGSYATAVAAARAYDTAVFYLRGRSARLNFPEEIPSFGLADGVDVGEHARDPAAAAAGGGGGCTLSAASIRKKAIEVGSRVDALQTGMVVPPPHHRERHRHHNHLPQLRVHAEEQQEEEEQKPQRPAWSGRVKNPDLNRAPSPESSDAE

SEQ ID NO: 12 - DNA, 702 bp, Zea mays
atgatgcagggcgagtaccggtcttccatcgaggagtcggctgcggccgctgttgccgcagccgcagccatggctcctctggcggccgcggcggtgaagatggaggcggagcaggcggcgatggcggcgccgcagctgggggcggcgcaccagcagacgcagccgcggcggcagtaccgcggcgtgcgcatgcgcaagtggggcaagtgggtggcggagatccgggagccgcacaagcgcacgcgcatctggctggggtcctacgccacggccgtggcggcggcgcgcgcctacgacacggccgtgttctacctgcgcgggcggtcggcgcggctcaacttccccgaggagatcccctcgttcgggctggcggatggcgtggacgtgggggagcacgcgcgcgacccggccgccgccgccgccggcggcggcggcggctgcacgctgtccgcggcgtccatacggaagaaggccatcgaggtggggtcccgcgtggacgcgctccagaccggcatggtggtcccgccgccgcaccaccgcgagcgccataggcaccacaaccacctgccgcagctgcgggtgcacgcggaggagcagcaggaggaagaggagcagaagccgcagcggcctgcgtggagcgggcgcgtcaagaacccggatctgaaccgtgcgccgagccccgagagctccgacgccgagtga

SEQ ID NO: 13 - protein, 193 aa, Zea mays
MERDATARAPLMPPPQPAPAPAAVLGAGVAVAVAPAAGNGRGGGGGGKQYRGVRMRKWGKWVAEIREPNKRSRIWLGSYSTAEAAARAYDTAVRAPLMLRGRAARLNFPDQLDGAQAAAADSTEGPRRDGAGAGLTAAAIRKKAAEVGARVDALHCGAGLAGGGTSPLLLSHRRRPKNPDLNREPTPDTSDDE

SEQ ID NO: 14 - DNA, 564 bp, Zea mays
atggagagggacgccacagccttctgcccgccgccccagcccgccccggctcctgcggcggtgctgggcgccggtgtcgcggtcgcggtcgcgccggcggcagggaacggccgcggcggcggcggcggagggaagcagtaccgcggcgtccggatgcgcaagtggggcaagtgggtggcggagatccgcgagcccaacaagcggtcgcggatctggctcgggtcctactccaccgccgaggccgccgcgcgcgcctacgacacggccgtcttctgcctgcgcggccgcgccgcgcggctcaacttccccgaccagctcgacggcgcgcaggcggcggcagcggacagcacggaggggccgcgcagggacggggccggggccgggctcaccgccgccgccatccgcaagaaggcggccgaggtcggggcccgcgtcgacgcgctccactgcggcgccggcctagctggtggcggcacctccccgctgctgctgtcccaccgccgccgccccaagaaccccgacctcaaccgggagcccaccccggacacctcggacgacgagtaa

SEQ ID NO: 15 - protein, 197 aa, Zea mays
MERDATARAPLMPPPQPAPAPASHAAVLGAGVAVAVAPAAGNGRGGGGGGKQYRGVRMRKWGKWVAEIREPNKRSRIWLGSYSTAEAAARAYDTAVRAPLMLRGRAARLNFPDQLDGAQAAAAADSTEGARRNGAGLTAAAIRKKAAEVGARVDALHCGAGAGLAGGGASPLLLSHRRRPKNPDLNREPTPDTSDDE

SEQ ID NO: 16 - DNA, 576 bp, Zea mays
atggagagggacgccacagccttctgcccgccgccccagcccgccccggctccggcgtcgcatgcggcggtgctgggcgccggtgtcgcggtcgcggtcgcgccggcggcagggaacggccgcggcggcggcggcggagggaagcagtaccgcggcgtccggatgcgcaagtggggcaagtgggtggcggagatccgcgagcccaacaagcggtcgcggatctggctcgggtcctactccaccgccgaggccgccgcgcgcgcctacgacacggccgtcttctgcctgcgcggccgcgccgcgcggctcaacttccccgaccagctcgacggcgcgcaggcggcggcggcggcggacagcacggagggggcgcgcaggaacggggccgggctcacggccgccgccatccgcaagaaggcggccgaggtcggggcccgcgtcgacgcgctccactgcggcgccggcgctggcctagcgggcggcggcgcctctccgctgctgctgtcccaccgccgtcgacccaagaaccccgacctcaaccgggagcccaccccggacacctcggacgacgagtaa

SEQ ID NO: 17 - protein, 183 aa, Zea mays
MEVDATARAPLMPPPQPAPAASPASKPPAPVLGAGGAVAPVAGHGRGGGRQYRGVRMRKWGKWVAEIREPNKRSRIWLGSYSTAVAAACAYDTAVRAPLMLRGRSARLNFPDQLDGAAVADNPEDGAGLTAAAIRKKAAEVGARVDALHCGGGTSPQPLSFHRRRPKNPDLNREPTPETSDDE

SEQ ID NO: 18 - DNA, 534 bp, Zea mays
atggaggtggacgccactgccttctgcccgccgccccagcccgcgcctgccgcctccccggcttcaaagccgcctgcgccggtgctgggcgccggtggagcggtcgcgccggtggcagggcacggccgcggcggagggaggcagtaccgcggcgtccggatgcgcaagtggggcaagtgggtggcggagatccgcgagccgaacaagcggtcgcggatctggctcgggtcctactccaccgccgtcgccgccgcgtgcgcctacgacacggccgtcttctgcctccgcggccgctccgcgcggctcaacttccccgaccagctcgacggcgcggcggtggccgacaaccccgaggacggcgcggggctcacagccgccgccatccgcaagaaggcggccgaggtcggcgcccgcgtcgacgcgctccactgcggcggcggcacctctccgcagccgctgtcgttccatcgccgccgccccaagaaccccgacctcaaccgggagccaaccccggaaacctcagacgacgagtaa

(19) United States
(12) Patent Application Publication
(10) Pub. No.: US 2018/0355370 A1
(43) Pub. Date: Dec. 13, 2018

(54) DREB REPRESSOR MODIFICATIONS AND METHODS TO INCREASE AGRONOMIC PERFORMANCE OF PLANTS

(71) Applicant: PIONEER HI-BRED INTERNATIONAL, INC., JOHNSTON, IA (US)

(72) Inventors: MARIA HRMOVA, FELIXSTOW (AU); AMRITHA AMALRAJ, MALVERN (AU); SERGIY LOPATO, MORPHETT VALE (AU)

(73) Assignee: PIONEER HI-BRED INTERNATIONAL, INC., JOHNSTON, IA (US)

(21) Appl. No.: 15/577,789

(22) PCT Filed: Jun. 9, 2016
(86) PCT No.: PCT/US16/36586

§ 371 Date: Nov. 29, 2017

Related U.S. Application Data
(60) Provisional application No. 62/173,613, filed on Jun. 10, 2015.

(51) Int. Cl. C12N 15/82; C07K 14/415

(52) U.S. Cl. CPC C12N 15/8273; C07K 14/415; C12N 15/8269; C12N 15/8261

(57) ABSTRACT

RAP2.1L without a functional EAR motif expression results in improved agronomic performance including various photosynthetic characteristics including drought and cold tolerance. Methods and compositions that affect yield and other agronomic characteristics in plants are disclosed.

### FIELD

The disclosure relates generally to the field of molecular biology.

### REFERENCE TO SEQUENCE LISTING SUBMITTED ELECTRONICALLY

The official copy of the sequence listing is submitted electronically via EFS-Web as an ASCII formatted sequence listing with a file named “RTS20250F_ST25.txt” created on Jun. 5, 2016 and having a size of 25 kilobytes and is filed concurrently with the specification. The sequence listing contained in this ASCII formatted document is part of the specification and is herein incorporated by reference in its entirety.

### BACKGROUND

Improving agronomic traits in crop plants is beneficial to farmers. Several factors influence crop yield. Abiotic stress is the primary cause of crop loss worldwide, causing average yield losses of more than 50% for major crops. Among the various abiotic stresses, drought is a major factor that limits crop productivity worldwide. Exposure of plants to a water-limiting environment during various developmental stages appears to activate various physiological and developmental changes. Molecular mechanisms of abiotic stress responses and the genetic regulatory networks of drought stress tolerance have been studied.

Plants are exposed to various stresses in their natural environment. One of the first plant responses to stress is a reorganization of the plant transcriptome. The Dehydration-Responsive Element Binding (DREB) proteins or C-Repeat Binding Factors (CBF) can positively or negatively regulate promoters of multiple drought- and cold-responsive genes by binding to Dehydration Responsive Element/C-repeat (DRE/CRT), through the APETALA2 (AP2) domain.

### SUMMARY

The present disclosure provides polynucleotides, related polypeptides and all conservatively modified variants of RAP2.1L that have been shown to affect agronomic parameters in crop plants. In an embodiment, RAP2.1L and variants modulate drought tolerance and one or more other agronomic characteristics of a plant. In an embodiment, plants overexpressing RAP2.1L and variants had increased drought tolerance and cold/frost tolerance.

Methods of improving an agronomic characteristic of a plant, the method includes modulating the expression of (i) a polynucleotide encoding an amino acid sequence comprising SEQ ID NOS: 1, 3, 5, 7, 9, 11, 13, 15 and 17 or an amino acid sequence that is at least 95% identical to one of SEQ ID NOS: 1, 3, 5, 7, 9, 11, 13, 15 and 17 (ii) a polynucleotide that hybridizes under stringent hybridization conditions to a fragment of polynucleotide comprising SEQ ID NOS: 2, 4, 6, 8, 10, 12, 14, 16 and 18, wherein the fragment comprises at least 100 contiguous nucleotides of SEQ ID NOS: 2, 4, 6, 8, 10, 12, 14, 16 and 18 (iii) a polynucleotide that encodes an amino acid sequence that is at least 90% identical to SEQ ID NOS: 1, 3, 5, 7, 9, 11, 13, 15 and 17, (iv) a polynucleotide encoding a polypeptide comprising one or more deletions or insertions or substitutions of amino acids compared to SEQ ID NO: 1 or 2.

In an embodiment, the expression of the polynucleotide encoding a polypeptide having at least 95% identity to SEQ ID NOS: 1, 3, 5, 7, 9, 11, 13, 15 and 17 is increased by transforming the plant with a recombinant polynucleotide operably linked to a heterologous promoter. In an embodiment, the expression of an endogenous polynucleotide encoding a polypeptide having at least 95% identity to SEQ ID NOS: 1, 3, 5, 7, 9, 11, 13, 15 and 17 is increased by upregulating a regulatory element operably associated with the endogenous polynucleotide.

In an embodiment, the agronomic characteristic is selected from the group consisting of wilting avoidance, improved photosynthetic performance, increased chlorophyll content, increased photosynthetic rate, improved stomatal conductance, carboxylation efficiency, an increase in grain size, an increase in grain weight, an increase in grain yield, an increase in grain filling rate, and an increase in biomass. The increase in agronomic characteristic is measured with respect to a control plant that does not exhibit elevated levels of RAP2.1Lm (or a variant or an ortholog/homolog thereof). In an embodiment, the agronomic performance is an increase in drought tolerance. In an embodiment, the grain weight is increased in relation to a control plant not having an increased expression of the polynucleotide.

In an embodiment, the plant is a monocot. In an embodiment, the plant is wheat, barley, rice or maize. In an embodiment, the plant is a dicot. In an embodiment, the plant is soybean or brassica.

In an embodiment, methods of improving yield of a plant include increasing the expression of a polynucleotide that encodes a polypeptide comprising the amino acid sequence selected from the group consisting of SEQ ID NOS: 1, 3, 5, 7, 9, 11, 13, 15 and 17 or an allelic variant thereof.

In an embodiment, methods of improving grain yield include the expression of a polynucleotide that encodes a polypeptide comprising the amino acid sequence of SEQ ID NOS: 1, 3, 5, 7, 9, 11, 13, 15 and 17 or a variant thereof.

In an embodiment, methods of marker assisted selection of a plant or identifying a native trait associated with increased yield, include:

a. performing marker-assisted selection of plants that have one or more variations in genomic regions encoding a protein comprising SEQ ID NOS: 1, 3, 5, 7, 9, 11, 13, 15 and 17 or a variant thereof or a regulatory sequence thereof; and

b. identifying the plant that exhibits higher yield.

In an embodiment, methods of identifying one or more alleles in a population of plants that are associated with increased grain yield includes:

a. evaluating in a population of plants for one or more allelic variations in (i) a genomic region, the genomic region encoding a polypeptide or (ii) the regulatory region controlling the expression of the polypeptide, wherein the polypeptide comprises the amino acid sequence of SEQ ID NOS: 1, 3, 5, 7, 9, 11, 13, 15 and 17 or a sequence that is 95% identical to SEQ ID NOS: 1, 3, 5, 7, 9, 11, 13, 15 and 17;

b. obtaining phenotypic values of increased yield for the one or more plants in the population;

c. associating the allelic variations in the genomic region with the phenotype; and

d. identifying the one or more alleles that are associated with increased yield.

In an embodiment, a recombinant expression cassette includes the polynucleotide that is operably linked to a regulatory element, wherein the expression cassette is functional in a plant cell. In an embodiment, a host cell includes the expression cassette. In an embodiment, a plant includes the recombinant expression cassette.

In an embodiment, a plant part includes a plant regulatory element that operably regulates the expression of a polynucleotide encoding a polypeptide comprising the amino acid sequence of SEQ ID NOS: 1, 3, 5, 7, 9, 11, 13, 15 and 17 or a variant or an ortholog thereof, wherein the regulatory element is heterologous to the polynucleotide.

In an embodiment, the polynucleotide that comprises a fragment of SEQ ID NO: 1, is sufficient to up-regulate the endogenous expression of the polynucleotide that encodes a polypeptide.

In an embodiment, the modulation of the expression is achieved through mutagenesis. In an embodiment, the modulation of the expression is achieved through microRNA mediated gene silencing. In an embodiment, the modulation of the expression is achieved through promoter-mediated gene suppression. In an embodiment, the modulation of the expression is achieved through targeted mutagenesis of an endogenous regulatory element.

In another aspect, the present disclosure relates to a recombinant expression cassette comprising a nucleic acid as described. Additionally, the present disclosure relates to a vector containing the recombinant expression cassette. Further, the vector containing the recombinant expression cassette can facilitate the transcription and translation of the nucleic acid in a host cell. The present disclosure also relates to the host cells able to express the polynucleotide of the present disclosure. A number of host cells could be used, such as but not limited to, microbial, mammalian, plant or insect.

In yet another embodiment, the present disclosure is directed to a plant or plant cells, containing the nucleic acids of the present disclosure. Preferred plants containing the polynucleotides of the present disclosure include but are not limited to maize, soybean, sunflower, sorghum, canola, wheat, alfalfa, cotton, rice, barley, tomato and millet. In another embodiment, the plant is a maize plant or plant cells. Another embodiment is the seeds from the nitrate uptake-associated polypeptide of the disclosure operably linked to a promoter that drives expression in the plant. The plants of the disclosure can have improved grain quality as compared to a control plant.

In an embodiment, a plant comprising increased expression of a polynucleotide encoding a polypeptide that is at least 95% identical to the amino acid sequence of SEQ ID NOS: 1, 7, 9, 11, 13, 15, or 17, wherein the polynucleotide is operably linked by a heterologous regulatory element. In an embodiment, the plant is a maize plant or a wheat plant or a rice plant.

### BRIEF DESCRIPTION OF THE DRAWINGS

FIG. 1 shows TaRAP2.1L and TaRAP2.1Lmut over-expression in wheat and barley. Frost survival rates of (A) control (WT) and three lines of barley transformed with pUbi-TaRAP2.1L, and (B) WT and four lines of wheat (cv. Bobwhite) transformed with pDhn8-TaRAP2.1L. Frost survival rates of WT and wheat transformed with pUbi-TaRAP2Lmut. Frost survival rates of (C) T1 lines and (D) T3 homozygous progeny of the same lines. Statistical data were calculated from 12 plants. In panels B, C and F, standard error bars are indicated. In panel E, asterisks represent significant differences compared to control plants at the 5% (P<0.05) significance level and were calculated by one-way ANOVA (GenStat 9.0).

FIG. 2 shows yield components and drought survival rates of control and wheat transformed with pUbi-TaRAP2Lmut. (A) Growth characteristics and yield components of T2 sub-lines grown in large containers under well-watered conditions or moderate drought conditions. (B) Survival rates for T3 sub-lines grown in pots, after re-watering from a severe drought event were recorded after six weeks. Statistical data were calculated from 12 to 16 plants. Values represent means±SE at P<0.05 that were calculated by one-way ANOVA (GenStat 9.0).

FIG. 3 shows phylogeny reconstruction of ERF, DREB, DEAR and RAP TFs from mono- and dicotyledonous plant species. The evolutionary relationships were inferred by using the Maximum Likelihood method based on the Jones, Taylor and Thornton matrix-based model. Branches with corresponding partitions are indicated. The distance scale length of 0.05 is indicated. Entries with EAR motifs, are marked with blue circles. The position of TaRAP2.1L in the tree is shown in bold. Two letter prefixes for sequence identifiers indicate species of origin: (Ta=Triticum aestivum; Hv=Hordeum vulgare; At=Arabidopsis thaliana; Zm=Zea mays, etc.). A full list of all entries is specified in Table S1.

FIG. 4 shows growth and yield components of control and wheat (T1) transformed with pUbi-TaRAP2Lmut. Growth characteristics and yield components were determined for plants grown in pots under well-watered conditions. Data obtained for null-segregants were not used. Statistical data were calculated from five to eight plants. Single asterisks represent significant difference compared to control plants at the 5% (P<0.05) significance level. Statistical analyses were performed by one-way ANOVA (GenStat 9.0).

### SEQUENCE LISTING

The sequence descriptions and Sequence Listing attached hereto comply with the rules governing nucleotide and/or amino acid sequence disclosures in patent applications as set forth in 37 C.F.R. § 1.821-1.825.

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984) which are herein incorporated by reference. The symbols and format used for nucleotide and amino acid sequence data comply with the rules set forth in 37 C.F.R. § 1.822.

TABLE 1
Sequence Listing

| Name | SEQ ID NO |
|---|---|
| TaRAP2.1L amino acid | 1 |
| TaRAP2.1L (DNA) | 2 |
| TaRAP2.1L1 (amino acid) | 3 |
| TaRAP2.1L1 (DNA) | 4 |
| TaDBP (amino acid) | 5 |
| TaDBP (DNA) | 6 |
| ZmRAP2_10L3 (amino acid) | 7 |
| ZmRAP2_10L3 (DNA) | 8 |
| ZmRAP2_10L (amino acid) | 9 |
| ZmRAP2_10L (DNA) | 10 |
| ZmRAP2_10L1 (amino acid) | 11 |
| ZmRAP2_10L1 (DNA) | 12 |
| ZmDREB_NP_0011 (amino acid) | 13 |
| ZmDREB_NP_0011 (DNA) | 14 |
| ZmDREB_AFW85670 (amino acid) | 15 |
| ZmDREB_AFW85670 (DNA) | 16 |
| ZmDREB_AFW75641 (amino acid) | 17 |
| ZmDREB_AFW75641 (DNA) | 18 |

### DETAILED DESCRIPTION

The contents of the priority provisional application 62/173,613 filed Jun. 10, 2015 is hereby incorporated by reference in its entirety.

A method of producing a seed, the method comprising: (a) crossing a first plant with a second plant, wherein at least one of the first plant and the second plant comprises a recombinant DNA construct, wherein the recombinant DNA construct comprises a polynucleotide operably linked to at least one heterologous regulatory element, wherein the polynucleotide encodes a polypeptide having an amino acid sequence of at least 70%, 75%, 80%, 85%, 90%, 91%, 92%, 93%, 94%, 95%, 96%, 97%, 98%, 99% or 100% sequence identity, based on the Clustal V or the Clustal W method of alignment, using the respective default parameters, when compared to SEQ ID NOS: 1, 3, 5, 7, 9, 11, 13, 15 and 17; and (b) selecting a seed of the crossing of step (a), wherein the seed comprises the recombinant DNA construct. A plant grown from the seed may exhibit at least one trait selected from the group consisting of: increased abiotic stress tolerance, increased yield, increased biomass, and altered root architecture, when compared to a control plant not comprising the recombinant DNA construct. The polypeptide may be over-expressed in at least one tissue of the plant, or during at least one condition of abiotic stress, or both. The plant may be selected from the group consisting of: maize, soybean, sunflower, sorghum, canola, wheat, alfalfa, cotton, rice, barley, millet, sugar cane and switchgrass.

A method of producing a plant that exhibits an increase in at least one trait selected from the group consisting of: increased abiotic stress tolerance, increased yield, increased biomass, and altered root architecture, wherein the method comprises growing a plant from a seed comprising a recombinant DNA construct, wherein the recombinant DNA construct comprises a polynucleotide operably linked to at least one heterologous regulatory element, wherein the polynucleotide encodes a polypeptide having an amino acid sequence of at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, based on the Clustal V or the Clustal W method of alignment, using the respective default parameters, when compared to SEQ ID NOS: 1, 3, 5, 7, 9, 11, 13, 15 and 17, wherein the plant exhibits at least one trait selected from the group consisting of: increased nitrogen stress tolerance, increased yield, increased biomass, and altered root architecture, when compared to a control plant not comprising the recombinant DNA construct. The polypeptide may be over-expressed in at least one tissue of the plant, or during at least one condition of abiotic stress, or both. The plant may be selected from the group consisting of: maize, soybean, sunflower, sorghum, canola, wheat, alfalfa, cotton, rice, barley, millet, sugar cane and switchgrass.

The role of DREB/CBF transcriptional activators in regulating plant responses to abiotic stresses is known. In addition to a large number of transcriptional activators, the DREB/CBF subfamily also contains a small group of factors with active repressor EAR motifs. In Arabidopsis these proteins have been designated as RAP2.1 and DEAR proteins (Dong and Liu, 2010 BMC Plant Biol 10, 47; Tsutsui et al., 2009 J Plant Res 122, 633-643). A wheat homologue of RAP2.1, TaRAP2.1L, and evaluated for increasing the stress tolerance and performance of wheat plants by altering function of the TaRAP2.1L gene product.

“RAP2.1Lm” as used in refers to a monocot RAP2.1 polypeptide that does not contain a functional EAR motif. These include RAP2.1 polypeptides with mutations or deletions or insertions to the EAR motif that render the EAR motif non-functional. For example, one or more amino acid changes to the motif that contains amino acids, -DLN--P motif render the Rap2.1 polypeptide without a functional EAR motif. These amino acid changes include substitutions, deletions, and insertions to one or more amino acids within or around the EAR motif.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS) and strand displacement amplification (SDA). See, e.g., Diagnostic Molecular Microbiology: Principles and Applications, Persing, et al., eds., American Society for Microbiology, Washington, D.C. (1993). The product of amplification is termed an amplicon.

It is understood, as those skilled in the art will appreciate, that the disclosure encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N terminal and C terminal portions of the polypeptide molecule would also not be expected to alter the activity of the polypeptide. Each of the proposed modifications is well within the routine skill in the art, as is determination of retention of biological activity of the encoded products.

The protein disclosed herein may also be a protein which comprises an amino acid sequence comprising deletion, substitution, insertion and/or addition of one or more amino acids in an amino acid sequence selected from the group consisting of SEQ ID NO: 1 or variants thereof. The substitution may be conservative, which means the replacement of a certain amino acid residue by another residue having similar physical and chemical characteristics. Non-limiting examples of conservative substitution include replacement between aliphatic group-containing amino acid residues such as Ile, Val, Leu or Ala, and replacement between polar residues such as Lys-Arg, Glu-Asp or Gln-Asn replacement.

Proteins derived by amino acid deletion, substitution, insertion and/or addition can be prepared when DNAs encoding their wild-type proteins are subjected to, for example, well-known site-directed mutagenesis (see, e.g., Nucleic Acid Research, Vol. 10, No. 20, p. 6487-6500, 1982, which is hereby incorporated by reference in its entirety). As used herein, the term “one or more amino acids” is intended to mean a possible number of amino acids which may be deleted, substituted, inserted and/or added by site-directed mutagenesis.

Site-directed mutagenesis may be accomplished, for example, as follows using a synthetic oligonucleotide primer that is complementary to single-stranded phage DNA to be mutated, except for having a specific mismatch (i.e., a desired mutation). Namely, the above synthetic oligonucleotide is used as a primer to cause synthesis of a complementary strand by phages, and the resulting duplex DNA is then used to transform host cells. The transformed bacterial culture is plated on agar, whereby plaques are allowed to form from phage-containing single cells. As a result, in theory, 50% of new colonies contain phages with the mutation as a single strand, while the remaining 50% have the original sequence. At a temperature which allows hybridization with DNA completely identical to one having the above desired mutation, but not with DNA having the original strand, the resulting plaques are allowed to hybridize with a synthetic probe labeled by kinase treatment. Subsequently, plaques hybridized with the probe are picked up and cultured for collection of their DNA.

Techniques for allowing deletion, substitution, insertion and/or addition of one or more amino acids in the amino acid sequences of biologically active peptides such as enzymes while retaining their activity include site-directed mutagenesis mentioned above, as well as other techniques such as those for treating a gene with a mutagen, and those in which a gene is selectively cleaved to remove, substitute, insert or add a selected nucleotide or nucleotides, and then ligated.

The protein disclosed herein may also be a protein which is encoded by a nucleic acid comprising a nucleotide sequence comprising deletion, substitution, insertion and/or addition of one or more nucleotides in a nucleotide sequence selected from the group consisting of sequences encoding SEQ ID NOS: 1, 3, 5, 7, 9, 11, 13, 15 and 17. Nucleotide deletion, substitution, insertion and/or addition may be accomplished by site-directed mutagenesis or other techniques as mentioned above.

The protein disclosed herein may also be a protein which is encoded by a nucleic acid comprising a nucleotide sequence hybridizable under stringent conditions with the complementary strand of a nucleotide sequence selected from the group consisting of sequences encoding SEQ ID NOS: 2, 4, 6, 8, 10, 12, 14, 16 and 18.

The term “under stringent conditions” means that two sequences hybridize under moderately or highly stringent conditions. More specifically, moderately stringent conditions can be readily determined by those having ordinary skill in the art, e.g., depending on the length of DNA. The basic conditions are set forth by Sambrook et al., Molecular Cloning: A Laboratory Manual, third edition, chapters 6 and 7, Cold Spring Harbor Laboratory Press, 2001 and include the use of a prewashing solution for nitrocellulose filters 5×SSC, 0.5% SDS, 1.0 mM EDTA (pH 8.0), hybridization conditions of about 50% formamide, 2×SSC to 6×SSC at about 40-50° C. (or other similar hybridization solutions, such as Stark's solution, in about 50% formamide at about 42° C.) and washing conditions of, for example, about 40-60° C., 0.5-6×SSC, 0.1% SDS. Preferably, moderately stringent conditions include hybridization (and washing) at about 50° C. and 6×SSC. Highly stringent conditions can also be readily determined by those skilled in the art, e.g., depending on the length of DNA.

Generally, such conditions include hybridization and/or washing at higher temperature and/or lower salt concentration (such as hybridization at about 65° C., 6×SSC to 0.2×SSC, preferably 6×SSC, more preferably 2×SSC, most preferably 0.2×SSC), compared to the moderately stringent conditions. For example, highly stringent conditions may include hybridization as defined above, and washing at approximately 65-68° C., 0.2×SSC, 0.1% SDS. SSPE (1×SSPE is 0.15 M NaCl, 10 mM NaH2PO4, and 1.25 mM EDTA, pH 7.4) can be substituted for SSC (1×SSC is 0.15 M NaCl and 15 mM sodium citrate) in the hybridization and washing buffers; washing is performed for 15 minutes after hybridization is completed.

It is also possible to use a commercially available hybridization kit which uses no radioactive substance as a probe. Specific examples include hybridization with an ECL direct labeling & detection system. Stringent conditions include, for example, hybridization at 42° C. for 4 hours using the hybridization buffer included in the kit, which is supplemented with 5% (w/v) Blocking reagent and 0.5 M NaCl, and washing twice in 0.4% SDS, 0.5×SSC at 55° C. for 20 minutes and once in 2×SSC at room temperature for 5 minutes.

By “encoding” or “encoded,” with respect to a specified nucleic acid, is meant comprising the information for translation into the specified protein. A nucleic acid encoding a protein may comprise non-translated sequences (e.g., introns) within translated regions of the nucleic acid or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal, and fungal mitochondria, the bacterium Mycoplasma capricolum (Yamao, et al., (1985) Proc. Natl. Acad. Sci. USA 82:2306-9) or the ciliate Macronucleus, may be used when the nucleic acid is expressed using these organisms.

When the nucleic acid is prepared or altered synthetically, advantage can be taken of known codon preferences of the intended host where the nucleic acid is to be expressed. For example, although nucleic acid sequences of the present disclosure may be expressed in both monocotyledonous and dicotyledonous plant species, sequences can be modified to account for the specific codon preferences and GC content preferences of monocotyledonous plants or dicotyledonous plants as these preferences have been shown to differ (Murray, et al., (1989) Nucleic Acids Res. 17:477-98 and herein incorporated by reference). Thus, the maize preferred codon for a particular amino acid might be derived from known gene sequences from maize. Maize codon usage for 28 genes from maize plants is listed in Table 4 of Murray, et al., supra.

As used herein, “heterologous” in reference to a nucleic acid is a nucleic acid that originates from a foreign species, or, if from the same species, is substantially modified from its native form in composition and/or genomic locus by deliberate human intervention. Heterologous may also indicate that a particular nucleic acid is foreign to its location in the genome as compared to its native location in the genome. For example, a promoter operably linked to a heterologous structural gene is from a species different from that from which the structural gene was derived or, if from the same species, one or both are substantially modified from their original form. A heterologous protein may originate from a foreign species or, if from the same species, is substantially modified from its original form by deliberate human intervention.

By “host cell” is meant a cell, which comprises a heterologous nucleic acid sequence of the disclosure, which contains a vector and supports the replication and/or expression of the expression vector. Host cells may be prokaryotic cells such as E. coli, or eukaryotic cells such as yeast, insect, plant, amphibian or mammalian cells. Preferably, host cells are monocotyledonous or dicotyledonous plant cells, including but not limited to maize, sorghum, sunflower, soybean, wheat, alfalfa, rice, cotton, canola, barley, millet and tomato. A particularly preferred monocotyledonous host cell is a maize host cell.

The term “hybridization complex” includes reference to a duplex nucleic acid structure formed by two single-stranded nucleic acid sequences selectively hybridized with each other.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon or transiently expressed (e.g., transfected mRNA).

The terms “isolated” refers to material, such as a nucleic acid or a protein, which is substantially or essentially free from components which normally accompany or interact with it as found in its naturally occurring environment. The isolated material optionally comprises material not found with the material in its natural environment. Nucleic acids, which are “isolated”, as defined herein, are also referred to as “heterologous” nucleic acids. Unless otherwise stated, the term “nitrate uptake-associated nucleic acid” means a nucleic acid comprising a polynucleotide (“nitrate uptake-associated polynucleotide”) encoding a full length or partial length nitrate uptake-associated polypeptide.

As used herein, “nucleic acid” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules, which comprise and substantially represent the entire transcribed fraction of a genome of a specified organism.

As used herein “operably linked” includes reference to a functional linkage between a first sequence, such as a promoter, and a second sequence, wherein the promoter sequence initiates and mediates transcription of the DNA corresponding to the second sequence. Generally, operably linked means that the nucleic acid sequences being linked are contiguous and, where necessary to join two protein coding regions, contiguous and in the same reading frame.

As used herein, the term “plant” includes reference to whole plants, plant organs (e.g., leaves, stems, roots, etc.), seeds and plant cells and progeny of same. Plant cell, as used herein includes, without limitation, seeds, suspension cultures, embryos, meristematic regions, callus tissue, leaves, roots, shoots, gametophytes, sporophytes, pollen and microspores. The class of plants, which can be used in the methods of the disclosure, is generally as broad as the class of higher plants amenable to transformation techniques, including both monocotyledonous and dicotyledonous plants including species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, Allium and Triticum. A particularly preferred plant is Zea mays.

As used herein, “polynucleotide” includes reference to a deoxyribopolynucleotide, ribopolynucleotide or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including inter alia, simple and complex cells.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells. Exemplary plant promoters include, but are not limited to, those that are obtained from plants, plant viruses and bacteria which comprise genes expressed in plant cells such Agrobacterium or Rhizobium. Examples are promoters that preferentially initiate transcription in certain tissues, such as leaves, roots, seeds, fibres, xylem vessels, tracheids or sclerenchyma. Such promoters are referred to as “tissue preferred.” A “cell type” specific promoter primarily drives expression in certain cell types in one or more organs, for example, vascular cells in roots or leaves. An “inducible” or “regulatable” promoter is a promoter, which is under environmental control. Examples of environmental conditions that may effect transcription by inducible promoters include anaerobic conditions or the presence of light. Another type of promoter is a developmentally regulated promoter, for example, a promoter that drives expression during pollen development. Tissue preferred, cell type specific, developmentally regulated and inducible promoters constitute the class of “non-constitutive” promoters. A “constitutive” promoter is a promoter, which is active under most environmental conditions. Suitable constitutive promoters include for example, Ubiquitin promoters, actin promoters, and GOS2 promoter (de Pater et al (1992), The Plant Journal, 2: 837-844).

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention or may have reduced or eliminated expression of a native gene. The term “recombinant” as used herein does not encompass the alteration of the cell or vector by naturally occurring events (e.g., spontaneous mutation, natural transformation/transduction/transposition) such as those occurring without deliberate human intervention.

As used herein, a “recombinant expression cassette” is a nucleic acid construct, generated recombinantly or synthetically, with a series of specified nucleic acid elements, which permit transcription of a particular nucleic acid in a target cell. The recombinant expression cassette can be incorporated into a plasmid, chromosome, mitochondrial DNA, plastid DNA, virus or nucleic acid fragment. Typically, the recombinant expression cassette portion of an expression vector includes, among other sequences, a nucleic acid to be transcribed and a promoter.

As used herein, “plant” includes reference to a plant, which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “ ” is used herein to include any cell, cell line, callus, tissue, plant part or plant, the genotype of which has been altered by the presence of heterologous nucleic acid including those s initially so altered as well as those created by sexual crosses or asexual propagation from the initial. The term “ ” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition or spontaneous mutation.

As used herein, “vector” includes reference to a nucleic acid used in transfection of a host cell and into which can be inserted a polynucleotide. Vectors are often replicons. Expression vectors permit transcription of a nucleic acid inserted therein.

The following terms are used to describe the sequence relationships between two or more nucleic acids or polynucleotides or polypeptides: (a) “reference sequence,” (b) “comparison window,” (c) “sequence identity,” (d) “percentage of sequence identity” and (e) “substantial identity.”

As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

As used herein, “comparison window” means includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence a gap penalty is typically introduced and is subtracted from the number of matches.

Methods of alignment of nucleotide and amino acid sequences for comparison are well known in the art. The local homology algorithm (BESTFIT) of Smith and Waterman, (1981) Adv. Appl. Math 2:482, may conduct optimal alignment of sequences for comparison; by the homology alignment algorithm (GAP) of Needleman and Wunsch, (1970) J. Mol. Biol. 48:443-53; by the search for similarity method (Tfasta and Fasta) of Pearson and Lipman, (1988) Proc. Natl. Acad. Sci. USA 85:2444; by computerized implementations of these algorithms, including, but not limited to: CLUSTAL in the PC/Gene program by Intelligenetics, Mountain View, Calif., GAP, BESTFIT, BLAST, FASTA and TFASTA in the Wisconsin Genetics Software Package, Version 8 (available from Genetics Computer Group (GCG® programs (Accelrys, Inc., San Diego, Calif.).). The CLUSTAL program is well described by Higgins and Sharp, (1988) Gene 73:237-44; Higgins and Sharp, (1989) CABIOS 5:151-3; Corpet, et al., (1988) Nucleic Acids Res. 16:10881-90; Huang, et al., (1992) Computer Applications in the Biosciences 8:155-65 and Pearson, et al., (1994) Meth. Mol. Biol. 24:307-31. The preferred program to use for optimal global alignment of multiple sequences is PileUp (Feng and Doolittle, (1987) J. Mol. Evol., 25:351-60 which is similar to the method described by Higgins and Sharp, (1989) CABIOS 5:151-53 and hereby incorporated by reference). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences and TBLASTX for nucleotide query sequences against nucleotide database sequences. See, Current Protocols in Molecular Biology, Chapter 19, Ausubel et al., eds., Greene Publishing and Wiley-Interscience, New York (1995).

As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17:149-63) and XNU (Claverie and States, (1993) Comput. Chem. 17:191-201) low-complexity filters can be employed alone or in combination.

As used herein, “sequence identity” or “identity” in the context of two nucleic acid or polypeptide sequences includes reference to the residues in the two sequences, which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences, which differ by such conservative substitutions, are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, (1988) Computer Applic. Biol. Sci. 4:11-17, e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has between 50-100% sequence identity, preferably at least 50% sequence identity, preferably at least 60% sequence identity, preferably at least 70%, more preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 55-100%, preferably at least 55%, preferably at least 60%, more preferably at least 70%, 80%, 90% and most preferably at least 95%.

### Orthologs and Paralogs

Homologous sequences as described above can comprise orthologous or paralogous sequences. Several different methods are known by those of skill in the art for identifying and defining these functionally homologous sequences. Three general methods for defining orthologs and paralogs are described; an ortholog, paralog or homolog may be identified by one or more of the methods described below.

Variant Nucleotide Sequences in the Non-Coding Regions

The nitrate uptake-associated nucleotide sequences are used to generate variant nucleotide sequences having the nucleotide sequence of the 5′-untranslated region, 3′-untranslated region or promoter region that is approximately 70%, 75%, 80%, 85%, 90% and 95% identical to the original nucleotide sequence of the corresponding SEQ ID NO: 1. These variants are then associated with natural variation in the germplasm for component traits related to grain quality and/or grain yield. The associated variants are used as marker haplotypes to select for the desirable traits.

Variant Amino Acid Sequences of RAP2.1Lm-Associated Polypeptides

Variant amino acid sequences of RAP2.1Lm-associated polypeptides are generated. In this example, one amino acid is altered. Specifically, the open reading frames are reviewed to determine the appropriate amino acid alteration. The selection of the amino acid to change is made by consulting the protein alignment (with the other orthologs and other gene family members from various species). An amino acid is selected that is deemed not to be under high selection pressure (not highly conserved) and which is rather easily substituted by an amino acid with similar chemical characteristics (i.e., similar functional side-chain). Using a protein alignment, an appropriate amino acid can be changed. Once the targeted amino acid is identified, the procedure outlined herein is followed. Variants having about 70%, 75%, 80%, 85%, 90% and 95% nucleic acid sequence identity are generated using this method. These variants are then associated with natural variation in the germplasm for component traits related to grain quality and/or grain yield. The associated variants are used as marker haplotypes to select for the desirable traits.

Synthetic Methods for Constructing Nucleic Acids

The isolated nucleic acids of the present disclosure can also be prepared by direct chemical synthesis by methods such as the phosphotriester method of Narang, et al., (1979) Meth. Enzymol. 68:90-9; the phosphodiester method of Brown, et al., (1979) Meth. Enzymol. 68:109-51; the diethylphosphoramidite method of Beaucage, et al., (1981) Tetra. Letts. 22(20):1859-62; the solid phase phosphoramidite triester method described by Beaucage, et al., supra, e.g., using an automated synthesizer, e.g., as described in Needham-VanDevanter, et al., (1984) Nucleic Acids Res. 12:6159-68 and the solid support method of U.S. Pat. No. 4,458,066. Chemical synthesis generally produces a single stranded oligonucleotide. This may be converted into double stranded DNA by hybridization with a complementary sequence or by polymerization with a DNA polymerase using the single strand as a template. One of skill will recognize that while chemical synthesis of DNA is limited to sequences of about 100 bases, longer sequences may be obtained by the ligation of shorter sequences.

### UTRs and Codon Preference

In general, translational efficiency has been found to be regulated by specific sequence elements in the 5′ non-coding or untranslated region (5′ UTR) of the RNA. Positive sequence motifs include translational initiation consensus sequences (Kozak, (1987) Nucleic Acids Res. 15:8125) and the 5<G> 7 methyl GpppG RNA cap structure (Drummond, et al., (1985) Nucleic Acids Res. 13:7375). Negative elements include stable intramolecular 5′ UTR stem-loop structures (Muesing, et al., (1987) Cell 48:691) and AUG sequences or short open reading frames preceded by an appropriate AUG in the 5′ UTR (Kozak, supra, Rao, et al., (1988) Mol. and Cell. Biol. 8:284). Accordingly, the present disclosure provides 5′ and/or 3′ UTR regions for modulation of translation of heterologous coding sequences.

### Plant Transformation Methods

Numerous methods for introducing foreign genes into plants are known and can be used to insert a nitrate uptake-associated polynucleotide into a plant host, including biological and physical plant transformation protocols. See, e.g., Miki, et al., “Procedure for Introducing Foreign DNA into Plants,” in Methods in Plant Molecular Biology and Biotechnology, Glick and Thompson, eds., CRC Press, Inc., Boca Raton, pp. 67-88 (1993). The methods chosen vary with the host plant, and include chemical transfection methods such as calcium phosphate, microorganism-mediated gene transfer such as Agrobacterium (Horsch et al., (1985) Science 227:1229-31), electroporation, microinjection and biolistic bombardment.

Expression cassettes and vectors and in vitro culture methods for plant cell or tissue transformation and regeneration of plants are known and available. See, e.g., Gruber et al., “Vectors for Plant Transformation,” in Methods in Plant Molecular Biology and Biotechnology, supra, pp. 89-119.

The isolated polynucleotides or polypeptides may be introduced into the plant by one or more techniques typically used for direct delivery into cells. Such protocols may vary depending on the type of organism, cell, plant or plant cell, i.e., monocot or dicot, targeted for gene modification. Suitable methods of transforming plant cells include microinjection (Crossway, et al., (1986) Biotechniques 4:320-334 and U.S. Pat. No. 6,300,543), electroporation (Riggs, et al., (1986) Proc. Natl. Acad. Sci. USA 83:5602-5606, direct gene transfer (Paszkowski, et al., (1984) EMBO J. 3:2717-2722) and ballistic particle acceleration (see, for example, Sanford, et al., U.S. Pat. No. 4,945,050; WO 91/10725 and McCabe, et al., (1988) Biotechnology 6:923-926). Also see, Tomes, et al., “Direct DNA Transfer into Intact Plant Cells Via Microprojectile Bombardment”. pp. 197-213 in Plant Cell, Tissue and Organ Culture, Fundamental Methods. eds. Gamborg and Phillips. Springer-Verlag Berlin Heidelberg New York, 1995; U.S. Pat. No. 5,736,369 (meristem); Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); WO 91/10725 (maize); Klein, et al., (1988) Plant Physiol. 91:440-444 (maize); Fromm, et al., (1990) Biotechnology 8:833-839 and Gordon-Kamm, et al., (1990) Plant Cell 2:603-618 (maize); Hooydaas-Van Slogteren and Hooykaas (1984) Nature (London) 311:763-764; Bytebierm, et al., (1987) Proc. Natl. Acad. Sci. USA 84:5345-5349 (Liliaceae); De Wet, et al., (1985) In The Experimental Manipulation of Ovule Tissues, ed. Chapman, et al., pp. 197-209. Longman, NY (pollen); Kaeppler, et al., (1990) Plant Cell Reports 9:415-418; and Kaeppler, et al., (1992) Theor. Appl. Genet. 84:560-566 (whisker-mediated transformation); U.S. Pat. No. 5,693,512 (sonication); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotech. 14:745-750; Agrobacterium mediated maize transformation (U.S. Pat. No. 5,981,840); silicon carbide whisker methods (Frame, et al., (1994) Plant J. 6:941-948); laser methods (Guo, et al., (1995) Physiologia Plantarum 93:19-24); sonication methods (Bao, et al., (1997) Ultrasound in Medicine & Biology 23:953-959; Finer and Finer, (2000) Lett Appl Microbiol. 30:406-10; Amoah, et al., (2001) J Exp Bot 52:1135-42); polyethylene glycol methods (Krens, et al., (1982) Nature 296:72-77); protoplasts of monocot and dicot cells can be transformed using electroporation (Fromm, et al., (1985) Proc. Natl. Acad. Sci. USA 82:5824-5828) and microinjection (Crossway, et al., (1986) Mol. Gen. Genet. 202:179-185), all of which are herein incorporated by reference.

### Agrobacterium-Mediated Transformation

The most widely utilized method for introducing an expression vector into plants is based on the natural transformation system of Agrobacterium. A. tumefaciens and A. rhizogenes are plant pathogenic soil bacteria, which genetically transform plant cells. The Ti and Ri plasmids of A. tumefaciens and A. rhizogenes, respectively, carry genes responsible for genetic transformation of plants. See, e.g., Kado, (1991) Crit. Rev. Plant Sci. 10:1. Descriptions of the Agrobacterium vector systems and methods for Agrobacterium-mediated gene transfer are provided in Gruber, et al., supra; Miki, et al., supra and Moloney, et al., (1989) Plant Cell Reports 8:238.

Similarly, the gene can be inserted into the T-DNA region of a Ti or Ri plasmid derived from A. tumefaciens or A. rhizogenes, respectively. Thus, expression cassettes can be constructed as above, using these plasmids. Many control sequences are known which when coupled to a heterologous coding sequence and transformed into a host organism show fidelity in gene expression with respect to tissue/organ specificity of the original coding sequence. See, e.g., Benfey and Chua, (1989) Science 244:174-81. Particularly suitable control sequences for use in these plasmids are promoters for constitutive leaf-specific expression of the gene in the various target plants. Other useful control sequences include a promoter and terminator from the nopaline synthase gene (NOS). The NOS promoter and terminator are present in the plasmid pARC2, available from the American Type Culture Collection and designated ATCC 67238. If such a system is used, the virulence (vir) gene from either the Ti or Ri plasmid must also be present, either along with the T-DNA portion or via a binary system where the vir gene is present on a separate vector. Such systems, vectors for use therein, and methods of transforming plant cells are described in U.S. Pat. No. 4,658,082; U.S. patent application Ser. No. 913,914, filed Oct. 1, 1986, as referenced in U.S. Pat. No. 5,262,306, issued Nov. 16, 1993 and Simpson, et al., (1986) Plant Mol. Biol. 6:403-15 (also referenced in the '306 patent), all incorporated by reference in their entirety.

Once transformed, these cells can be used to regenerate plants. For example, whole plants can be infected with these vectors by wounding the plant and then introducing the vector into the wound site. Any part of the plant can be wounded, including leaves, stems and roots. Alternatively, plant tissue, in the form of an explant, such as cotyledonary tissue or leaf disks, can be inoculated with these vectors, and cultured under conditions, which promote plant regeneration. Roots or shoots transformed by inoculation of plant tissue with A. rhizogenes or A. tumefaciens, containing the gene coding for the fumonisin degradation enzyme, can be used as a source of plant tissue to regenerate fumonisin-resistant plants, either via somatic embryogenesis or organogenesis. Examples of such methods for regenerating plant tissue are disclosed in Shahin, (1985) Theor. Appl. Genet. 69:235-40; U.S. Pat. No. 4,658,082; Simpson, et al., supra; and U.S. patent application Ser. Nos. 913,913 and 913,914, both filed Oct. 1, 1986, as referenced in U.S. Pat. No. 5,262,306, issued Nov. 16, 1993, the entire disclosures therein incorporated herein by reference.

### Direct Gene Transfer

Despite the fact that the host range for Agrobacterium-mediated transformation is broad, some major cereal crop species and gymnosperms have generally been recalcitrant to this mode of gene transfer, even though some success has recently been achieved in rice (Hiei, et al., (1994) The Plant Journal 6:271-82). Several methods of plant transformation, collectively referred to as direct gene transfer, have been developed as an alternative to Agrobacterium-mediated transformation.

A generally applicable method of plant transformation is microprojectile-mediated transformation, where DNA is carried on the surface of microprojectiles measuring about 1 to 4 μm. The expression vector is introduced into plant tissues with a biolistic device that accelerates the microprojectiles to speeds of 300 to 600 m/s which is sufficient to penetrate the plant cell walls and membranes (Sanford, et al., (1987) Part. Sci. Technol. 5:27; Sanford, (1988) Trends Biotech 6:299; Sanford, (1990) Physiol. Plant 79:206 and Klein, et al., (1992) Biotechnology 10:268).

Another method for physical delivery of DNA to plants is sonication of target cells as described in Zang, et al., (1991) BioTechnology 9:996. Alternatively, liposome or spheroplast fusions have been used to introduce expression vectors into plants. See, e.g., Deshayes, et al., (1985) EMBO J. 4:2731 and Christou, et al., (1987) Proc. Natl. Acad. Sci. USA 84:3962. Direct uptake of DNA into protoplasts using CaCl₂) precipitation, polyvinyl alcohol or poly-L-ornithine has also been reported. See, e.g., Hain, et al., (1985) Mol. Gen. Genet. 199:161 and Draper, et al., (1982) Plant Cell Physiol. 23:451.

Electroporation of protoplasts and whole cells and tissues has also been described. See, e.g., Donn, et al., (1990) Abstracts of the VIIth Int'l. Congress on Plant Cell and Tissue Culture IAPTC, A2-38, p. 53; D'Halluin, et al., (1992) Plant Cell 4:1495-505 and Spencer, et al., (1994) Plant Mol. Biol. 24:51-61.

1. Polynucleotide-Based Methods:

In some embodiments of the present disclosure, a plant is transformed with an expression cassette that is capable of expressing a polynucleotide that inhibits the expression of RAP2.1Lm of the disclosure. The term “expression” as used herein refers to the biosynthesis of a gene product, including the transcription and/or translation of said gene product. For example, for the purposes of the present disclosure, an expression cassette capable of expressing a polynucleotide that inhibits the expression of at least one nitrate uptake-associated polypeptide is an expression cassette capable of producing an RNA molecule that inhibits the transcription and/or translation of at least one nitrate uptake-associated polypeptide of the disclosure. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide.

Examples of polynucleotides that inhibit the expression of RAP2.1Lm are given below.

i. Sense Suppression/Cosuppression

In some embodiments of the disclosure, inhibition of the expression of RAP2.1Lm may be obtained by sense suppression or cosuppression. For cosuppression, an expression cassette is designed to express an RNA molecule corresponding to all or part of a messenger RNA encoding RAP2.1Lm in the “sense” orientation. Over expression of the RNA molecule can result in reduced expression of the native gene. Accordingly, multiple plant lines transformed with the cosuppression expression cassette are screened to identify those that show the greatest inhibition of nitrate uptake-associated polypeptide expression.

The polynucleotide used for cosuppression may correspond to all or part of the sequence encoding the nitrate uptake-associated polypeptide, all or part of the 5′ and/or 3′ untranslated region of RAP2.1Lm transcript or all or part of both the coding sequence and the untranslated regions of a transcript encoding RAP2.1Lm. In some embodiments where the polynucleotide comprises all or part of the coding region for the nitrate uptake-associated polypeptide, the expression cassette is designed to eliminate the start codon of the polynucleotide so that no protein product will be translated.

Cosuppression may be used to inhibit the expression of plant genes to produce plants having undetectable protein levels for the proteins encoded by these genes. See, for example, Broin, et al., (2002) Plant Cell 14:1417-1432. Cosuppression may also be used to inhibit the expression of multiple proteins in the same plant. See, for example, U.S. Pat. No. 5,942,657. Methods for using cosuppression to inhibit the expression of endogenous genes in plants are described in Flavell, et al., (1994) Proc. Natl. Acad. Sci. USA 91:3490-3496; Jorgensen, et al., (1996) Plant Mol. Biol. 31:957-973; Johansen and Carrington, (2001) Plant Physiol. 126:930-938; Broin, et al., (2002) Plant Cell 14:1417-1432; Stoutjesdijk, et al., (2002) Plant Physiol. 129:1723-1731; Yu, et al., (2003) Phytochemistry 63:753-763 and U.S. Pat. Nos. 5,034,323, 5,283,184 and 5,942,657, each of which is herein incorporated by reference. The efficiency of cosuppression may be increased by including a poly-dT region in the expression cassette at a position 3′ to the sense sequence and 5′ of the polyadenylation signal. See, US Patent Publication Number 2002/0048814, herein incorporated by reference. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See, U.S. Pat. Nos. 5,283,184 and 5,034,323, herein incorporated by reference.

ii. Antisense Suppression

In some embodiments of the disclosure, inhibition of the expression of the nitrate uptake-associated polypeptide may be obtained by antisense suppression. For antisense suppression, the expression cassette is designed to express an RNA molecule complementary to all or part of a messenger RNA encoding the nitrate uptake-associated polypeptide. Over expression of the antisense RNA molecule can result in reduced expression of the native gene. Accordingly, multiple plant lines transformed with the antisense suppression expression cassette are screened to identify those that show the greatest inhibition of nitrate uptake-associated polypeptide expression.

iii. Double-Stranded RNA Interference

In some embodiments of the disclosure, inhibition of the expression of RAP2.1Lm may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules can be accomplished by designing the expression cassette to comprise both a sense sequence and an antisense sequence. Alternatively, separate expression cassettes may be used for the sense and antisense sequences. Methods for using dsRNA interference to inhibit the expression of endogenous plant genes are described in Waterhouse, et al., (1998) Proc. Natl. Acad. Sci. USA 95:13959-13964, Liu, et al., (2002) Plant Physiol. 129:1732-1743 and WO 99/49029, WO 99/53050, WO 99/61631 and WO 00/49035, each of which is herein incorporated by reference.

iv. Hairpin RNA Interference and Intron-Containing Hairpin RNA Interference

In some embodiments of the disclosure, inhibition of the expression of RAP2.1Lm may be obtained by hairpin RNA (hpRNA) interference or intron-containing hairpin RNA (ihpRNA) interference. These methods are highly efficient at inhibiting the expression of endogenous genes. See, Waterhouse and Helliwell, (2003) Nat. Rev. Genet. 4:29-38 and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited and an antisense sequence that is fully or partially complementary to the sense sequence. Alternatively, the base-paired stem region may correspond to a portion of a promoter sequence controlling expression of the gene to be inhibited. Thus, the base-paired stem region of the molecule generally determines the specificity of the RNA interference. hpRNA molecules are highly efficient at inhibiting the expression of endogenous genes and the RNA interference they induce is inherited by subsequent generations of plants. See, for example, Chuang and Meyerowitz, (2000) Proc. Natl. Acad. Sci. USA 97:4985-4990; Stoutjesdijk, et al., (2002) Plant Physiol. 129:1723-1731 and Waterhouse and Helliwell, (2003) Nat. Rev. Genet. 4:29-38. Methods for using hpRNA interference to inhibit or silence the expression of genes are described, for example, in Chuang and Meyerowitz, (2000) Proc. Natl. Acad. Sci. USA 97:4985-4990; Stoutjesdijk, et al., (2002) Plant Physiol. 129:1723-1731; Waterhouse and Helliwell, (2003) Nat. Rev. Genet. 4:29-38; Pandolfini et al., BMC Biotechnology 3:7, and US Patent Application Publication Number 2003/0175965, each of which is herein incorporated by reference. A transient assay for the efficiency of hpRNA constructs to silence gene expression in vivo has been described by Panstruga, et al., (2003) Mol. Biol. Rep. 30:135-140, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith, et al., (2000) Nature 407:319-320. In fact, Smith, et al., show 100% suppression of endogenous gene expression using ihpRNA-mediated interference. Methods for using ihpRNA interference to inhibit the expression of endogenous plant genes are described, for example, in Smith, et al., (2000) Nature 407:319-320; Wesley, et al., (2001) Plant J. 27:581-590; Wang and Waterhouse, (2001) Curr. Opin. Plant Biol. 5:146-150; Waterhouse and Helliwell, (2003) Nat. Rev. Genet. 4:29-38; Helliwell and Waterhouse, (2003) Methods 30:289-295 and US Patent Application Publication Number 2003/0180945, each of which is herein incorporated by reference.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, WO 02/00904; Mette, et al., (2000) EMBO J 19:5194-5201; Matzke, et al., (2001) Curr. Opin. Genet. Devel. 11:221-227; Scheid, et al., (2002) Proc. Natl. Acad. Sci., USA 99:13659-13662; Aufsaftz, et al., (2002) Proc. Natl. Acad. Sci. 99(4):16499-16506; Sijen, et al., Curr. Biol. (2001) 11:436-440), herein incorporated by reference.

v. Amplicon-Mediated Interference

Amplicon expression cassettes comprise a plant virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for the nitrate uptake-associated polypeptide). Methods of using amplicons to inhibit the expression of endogenous plant genes are described, for example, in Angell and Baulcombe, (1997) EMBO J. 16:3675-3684, Angell and Baulcombe, (1999) Plant J. 20:357-362 and U.S. Pat. No. 6,646,805, each of which is herein incorporated by reference.

vi. Small Interfering RNA or Micro RNA

In some embodiments of the disclosure, inhibition of the expression of RAP2.1Lm may be obtained by RNA interference by expression of a gene encoding a micro RNA (miRNA). miRNAs are regulatory agents consisting of about 22 ribonucleotides. miRNA are highly efficient at inhibiting the expression of endogenous genes. See, for example Javier, et al., (2003) Nature 425:257-263, herein incorporated by reference.

For miRNA interference, the expression cassette is designed to express an RNA molecule that is modeled on an endogenous miRNA gene. The miRNA gene encodes an RNA that forms a hairpin structure containing a 22-nucleotide sequence that is complementary to another endogenous gene (target sequence). For suppression of nitrate uptake-associated expression, the 22-nucleotide sequence is selected from a nitrate uptake-associated transcript sequence and contains 22 nucleotides of said nitrate uptake-associated sequence in sense orientation and 21 nucleotides of a corresponding antisense sequence that is complementary to the sense sequence. miRNA molecules are highly efficient at inhibiting the expression of endogenous genes and the RNA interference they induce is inherited by subsequent generations of plants.

2. Polypeptide-Based Inhibition of Gene Expression

In one embodiment, the polynucleotide encodes a zinc finger protein that binds to a gene encoding RAP2.1Lm, resulting in reduced expression or activity of the gene. In particular embodiments, the zinc finger protein binds to a regulatory region of a nitrate uptake-associated gene. In other embodiments, the zinc finger protein binds to a messenger RNA encoding RAP2.1Lm and prevents its translation. Methods of selecting sites for targeting by zinc finger proteins have been described, for example, in U.S. Pat. No. 6,453,242 and methods for using zinc finger proteins to inhibit the expression of genes in plants are described, for example, in US. Patent Application Publication Number 2003/0037355, each of which is herein incorporated by reference.

ii. Mutant Plants with Reduced Activity

Additional methods for decreasing or eliminating the expression of endogenous genes in plants are also known in the art and can be similarly applied to the instant disclosure. These methods include other forms of mutagenesis, such as ethyl methanesulfonate-induced mutagenesis, deletion mutagenesis, and fast neutron deletion mutagenesis used in a reverse genetics sense (with PCR) to identify plant lines in which the endogenous gene has been deleted. For examples of these methods see, Ohshima, et al., (1998) Virology 243:472-481; Okubara, et al., (1994) Genetics 137:867-874 and Quesada, et al., (2000) Genetics 154:421-436, each of which is herein incorporated by reference. In addition, a fast and automatable method for screening for chemically induced mutations, TILLING (Targeting Induced Local Lesions In Genomes), using denaturing HPLC or selective endonuclease digestion of selected PCR products is also applicable to the instant disclosure. See, McCallum, et al., (2000) Nat. Biotechnol. 18:455-457, herein incorporated by reference.

Mutations that impact gene expression or that interfere with the function (enhanced nitrogen utilization activity) of the encoded protein are well known in the art. Insertional mutations in gene exons usually result in null-mutants. Mutations in conserved residues are particularly effective in inhibiting the activity of the encoded protein. Conserved residues of plant nitrate uptake-associated polypeptides suitable for mutagenesis with the goal to eliminate nitrate uptake-associated activity have been described. Such mutants can be isolated according to well-known procedures, and mutations in different nitrate uptake-associated loci can be stacked by genetic crossing. See, for example, Gruis, et al., (2002) Plant Cell 14:2863-2882.

In another embodiment of this disclosure, dominant mutants can be used to trigger RNA silencing due to gene inversion and recombination of a duplicated gene locus. See, for example, Kusaba, et al., (2003) Plant Cell 15:1455-1467.

The disclosure encompasses additional methods for reducing or eliminating the activity of one or more nitrate uptake-associated polypeptide. Examples of other methods for altering or mutating a genomic nucleotide sequence in a plant are known in the art and include, but are not limited to, the use of RNA:DNA vectors, RNA:DNA mutational vectors, RNA:DNA repair vectors, mixed-duplex oligonucleotides, self-complementary RNA:DNA oligonucleotides and recombinogenic oligonucleobases. Such vectors and methods of use are known in the art. See, for example, U.S. Pat. Nos. 5,565,350; 5,731,181; 5,756,325; 5,760,012; 5,795,972 and 5,871,984, each of which are herein incorporated by reference. See also, WO 98/49350, WO 99/07865, WO 99/25821 and Beetham, et al., (1999) Proc. Natl. Acad. Sci. USA 96:8774-8778, each of which is herein incorporated by reference.

vi. Modulating Reproductive Tissue Development

Methods for modulating reproductive tissue development are provided. In one embodiment, methods are provided to modulate floral development in a plant. By “modulating floral development” is intended any alteration in a structure of a plant's reproductive tissue as compared to a control plant in which the activity or level of the nitrate uptake-associated polypeptide has not been modulated. “Modulating floral development” further includes any alteration in the timing of the development of a plant's reproductive tissue (i.e., a delayed or an accelerated timing of floral development) when compared to a control plant in which the activity or level of the nitrate uptake-associated polypeptide has not been modulated. Macroscopic alterations may include changes in size, shape, number, or location of reproductive organs, the developmental time period that these structures form or the ability to maintain or proceed through the flowering process in times of environmental stress. Microscopic alterations may include changes to the types or shapes of cells that make up the reproductive organs.

In general, methods to modify or alter the host endogenous genomic DNA are available. This includes altering the host native DNA sequence or a pre-existing sequence including regulatory elements, coding and non-coding sequences. These methods are also useful in targeting nucleic acids to pre-engineered target recognition sequences in the genome. As an example, the genetically modified cell or plant described herein, is generated using “custom” or engineered endonucleases such as meganucleases produced to modify plant genomes (see e.g., WO 2009/114321; Gao et al. (2010) Plant Journal 1:176-187). Another site-directed engineering is through the use of zinc finger domain recognition coupled with the restriction properties of restriction enzyme. See e.g., Urnov, et al., (2010) Nat Rev Genet. 11(9):636-46; Shukla, et al., (2009) Nature 459 (7245):437-41. A transcription activator-like (TAL) effector-DNA modifying enzyme (TALE or TALEN) is also used to engineer changes in plant genome. See e.g., US20110145940, Cermak et al., (2011) Nucleic Acids Res. 39(12) and Boch et al., (2009), Science 326(5959): 1509-12. Site-specific modification of plant genomes can also be performed using the bacterial type II CRISPR (clustered regularly interspaced short palindromic repeats)/Cas (CRISPR-associated) system. See e.g., Belhaj et al., (2013), Plant Methods 9: 39; The CRISPR/Cas system allows targeted cleavage of genomic DNA guided by a customizable small noncoding RNA. Based on the disclosure of the RAP2.1Lm coding sequences, polypeptide sequences of the orthologs/homologs and the genomic DNA sequences, site-directed mutagenesis can be readily performed to generate plants expressing a higher level of the endogenous RAP2.1Lm polypeptide or an ortholog thereof.

Antibodies to a RAP2.1Lm polypeptide disclosed herein or the embodiments or to variants or fragments thereof are also encompassed. The antibodies of the disclosure include polyclonal and monoclonal antibodies as well as fragments thereof which retain their ability to bind to RAP2.1Lm polypeptide disclosed herein. An antibody, monoclonal antibody or fragment thereof is said to be capable of binding a molecule if it is capable of specifically reacting with the molecule to thereby bind the molecule to the antibody, monoclonal antibody or fragment thereof. The term “antibody” (Ab) or “monoclonal antibody” (Mab) is meant to include intact molecules as well as fragments or binding regions or domains thereof (such as, for example, Fab and F(ab)₂ fragments) which are capable of binding hapten. Such fragments are typically produced by proteolytic cleavage, such as papain or pepsin. Alternatively, hapten-binding fragments can be produced through the application of recombinant DNA technology or through synthetic chemistry. Methods for the preparation of the antibodies of the present disclosure are generally known in the art. For example, see, Antibodies, A Laboratory Manual, Ed Harlow and David Lane (eds.) Cold Spring Harbor Laboratory, N.Y. (1988), as well as the references cited therein. Standard reference works setting forth the general principles of immunology include: Klein, J. Immunology: The Science of Cell-Noncell Discrimination, John Wiley & Sons, N.Y. (1982); Dennett, et al., Monoclonal Antibodies, Hybridoma: A New Dimension in Biological Analyses, Plenum Press, N.Y. (1980) and Campbell, “Monoclonal Antibody Technology,” In Laboratory Techniques in Biochemistry and Molecular Biology, Vol. 13, Burdon, et al., (eds.), Elsevier, Amsterdam (1984). See also, U.S. Pat. Nos. 4,196,265; 4,609,893; 4,713,325; 4,714,681; 4,716,111; 4,716,117 and 4,720,459. PtIP-50 polypeptide or PtIP-65 polypeptide antibodies or antigen-binding portions thereof can be produced by a variety of techniques, including conventional monoclonal antibody methodology, for example the standard somatic cell hybridization technique of Kohler and Milstein, (1975) Nature 256:495. Other techniques for producing monoclonal antibody can also be employed such as viral or oncogenic transformation of B lymphocytes. An animal system for preparing hybridomas is a murine system. Immunization protocols and techniques for isolation of immunized splenocytes for fusion are known in the art. Fusion partners (e.g., murine myeloma cells) and fusion procedures are also known. The antibody and monoclonal antibodies of the disclosure can be prepared by utilizing a RAP2.1Lm polypeptide disclosed herein as antigens.

A kit for detecting the presence of a RAP2.1Lm polypeptide disclosed herein or detecting the presence of a nucleotide sequence encoding a RAP2.1Lm polypeptide disclosed herein, in a sample is provided. In one embodiment, the kit provides antibody-based reagents for detecting the presence of a RAP2.1Lm polypeptide disclosed herein in a tissue sample. In another embodiment, the kit provides labeled nucleic acid probes useful for detecting the presence of one or more polynucleotides encoding RAP2.1Lm polypeptide disclosed herein. The kit is provided along with appropriate reagents and controls for carrying out a detection method, as well as instructions for use of the kit.

As discussed above, one of skill will recognize the appropriate promoter to use to modulate floral development of the plant. Exemplary promoters for this embodiment include constitutive promoters, inducible promoters, shoot-preferred promoters and inflorescence-preferred promoters.

Genes of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases and those involved in housekeeping, such as heat shock proteins. More specific categories of transgenes, for example, include genes encoding important traits for agronomics, insect resistance, disease resistance, herbicide resistance, sterility, grain characteristics and commercial products. Genes of interest include, generally, those involved in oil, starch, carbohydrate, or nutrient metabolism as well as those affecting kernel size, sucrose loading, and the like.

In certain embodiments the nucleic acid sequences of the present disclosure can be used in combination (“stacked”) with other polynucleotide sequences of interest in order to create plants with a desired phenotype.

This disclosure can be better understood by reference to the following non-limiting examples. It will be appreciated by those skilled in the art that other embodiments of the disclosure may be practiced without departing from the spirit and the scope of the disclosure as herein disclosed and claimed.

### EXAMPLES

### Example 1—TaRAP2.1L Expression is Induced by ABA and Abiotic Stresses

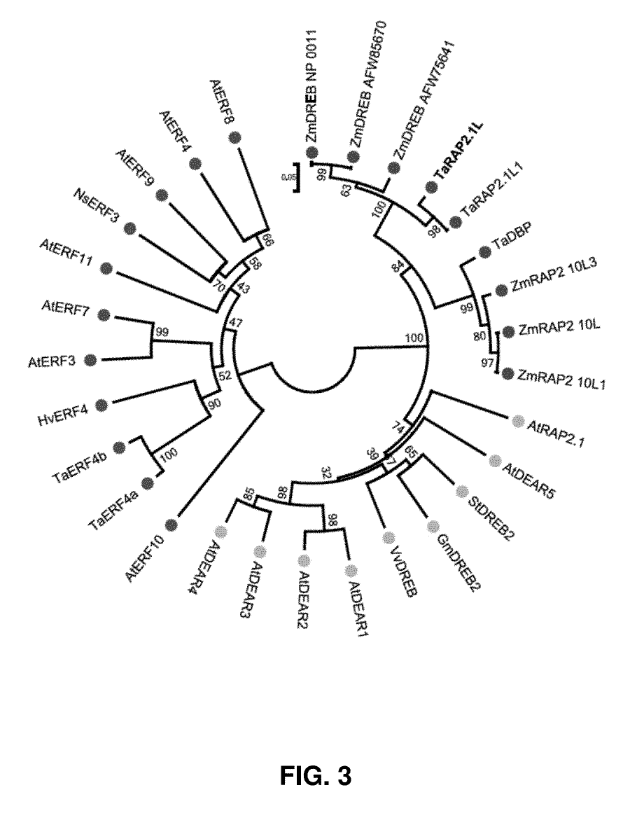

The full length cDNA clone encoding a wheat homologue of the Arabidopsis RAP2.1 protein was isolated from a cDNA library prepared from spikes and flag leaves of the drought-tolerant wheat cultivar RAC875, subjected to drought and heat stresses, using an optimised Y1H screening procedure. A DNA sequence containing four repeats of a core DRE cis-element from the Arabidopsis Rd29A promoter was used as bait DNA. The isolated RAP2.1-like gene, designated TaRAP2.1L (Ta is for Triticum aestivum L.) encodes a 184-residue protein with a domain structure similar to Arabidopsis RAP2.1, featuring the AP2 domain and two other conservative motifs, one of which is an EAR motif. The evolutionary relationship of 29 members of the EAR-containing AP2 domain TFs from mono- and dicotyledonous species was inferred by using the Maximum Likelihood method based on the Jones, Taylor and Thornton matrix-based model (FIG. 3). The tree demonstrated that one of the characterised proteins with the EAR motif, RAP2.1 from Arabidopsis, was the closest homologue of TaRAP2.1 and that the RAP2.1 clade was grouped with DREB proteins rather than with ERF proteins (FIG. 3, entries marked with blue circles). In the EST databases of T. aestivum cv. Chinese Spring, a close homologue (Acc. AK336006) was found, probably a homeologue of TaRAP2.1L, which was designated as TaRAP2.1L1 (FIG. 3).

Quantitative RT-PCR (Q-PCR) analyses of TaRAP2.1L expression revealed transcripts of TaRAP2.1L in all tested tissues with predominant expression in leaves, floral tissues and grain, and particularly strong expression in developed endosperm of a desiccating grain. Treatment of hydroponically-grown seedlings with elevated concentrations (200 μM) of ABA led to a 2.5-fold increase in TaRAP2.1L transcript numbers during 4 h of treatment. Expression levels of TaRAP2.1L were up-regulated four- to six-fold by drought and cold (at constant 4° C.). Expression was more responsive to a low temperature treatment than to slowly developing drought. Expression levels of TaRAP2.1L in both cases started to return to un-stressed levels before re-watering or temperature increase to 18° C. In contrast to TaDREB2, which was strongly activated by wounding in leaves, TaRAP2.1L was down-regulated about two-fold during the first 7 h after mechanical wounding, and 24 h after wounding the transcript number of TaRAP2.1L returned to the levels of an unstressed plant.

Expression of both TaRAP2.1L (DREB repressor) and TaDREB3 (DREB activator) was induced by rapid dehydration of detached leaves. TaRAP2.1L reached a maximal level of expression earlier than TaDREB3 and its up-regulation was shorter in time. TaCor39 was used as a dehydration-responsive reference gene.

Isolation of TaRAP2.1L

Four independent clones containing the full-length coding regions of TaRAP2.1L cDNA were isolated in a yeast one-hybrid (Y1H) screen of a cDNA library (WHSL) prepared from spikes and leaves of a drought-tolerant wheat cultivar (Triticum aestivum cv. RAC785), subjected to drought and heat stress. A broad Y2H cDNA library produced from drought tolerant wheat (cv. RAC875) plants subjected to heat (37° C.) and several stages (strength) of drought, until strong wilting. Flag leaves and spikes were collected at different stages of development starting with spikes at several days before flowering and finishing with spikes at about 10 days after flowering. RNA from leaves and spikes was isolated independently and mixed in equal proportions. RNA was isolated from the whole spike, including grain.

TaRAP2.1L cDNA was isolated using four consecutive repeats of DRE (4×, the core element is underlined) as a bait.

### Example 2—Domain Organisation of TaRAP2.1L

Independent analyses using SMART (Letunic et al., 2012, Nucleic Acids Res 40, D302-305), ProDom (Bru et al., 2005, Nucleic Acids Res 33, D212-215) and SBASE (Vlahovicek et al., 2002, Nucleic Acids Res 30, 273-275) of 29 selected members of AP2-containing TF proteins indicated the presence of at least two domains. The sequences analysed with ProDom showed that AP2 domains contained approximately 62 residues in all investigated proteins, although their precise dispositions within the full-length sequences differed, depending on the lengths of the unstructured N-terminal regions. The structural sequence alignment of 30 sequences provided information about the conservation of amino acid residues, and it was of note that all sequences contained the EAR signature motif that was positioned at their C-terminal ends. In the EAR signature motifs, most of the sequences contained Pro at the first position. Sequences without a Pro at this position carried a hydrophobic Leu, Val or Phe. The two other variable residues of the motif were less well conserved.

### Example 3—DNA Binding Properties of WT and Mutated TaRAP2.1L Proteins and the Functional Relationship to Abiotic Stress Response

Following identification of the repressor motif EAR at the C-terminus of TaRAP2.1L, the DNA binding specificity of the WT and TaRAP2.1Lmut proteins was investigated, carrying mutations in the EAR motif. TaRAP2.1Lmut was generated by replacing the four key residues of the EAR motif (-DLN--P) for Ala (-AAA--A). Two GAL4 binding domain (BD) fusion proteins were used in the yeast activation assay. The activation assay demonstrated that in contrast to BD-TaDREB3 (positive control), which activates a yeast reporter gene, BD-TaRAP2.1L had no trans-activation properties. The mutations in the EAR motif did not convert BD-TaRAP2.1Lmut to a transcriptional activator in yeast. Further, both Y1H and EMSA assays confirmed that the TaRAP2.1L protein had an unusual binding specificity. It could bind not only cis-elements DRE and CRT, which induce responses of plants to abiotic stresses and are recognised by all studied DREB/CBF proteins, but TaRAP2.1L also interacted with a GCC-box that is used by ERF proteins in response to biotic stresses. The data also showed that Ala mutations introduced in the EAR motif did not change the DNA-binding specificity of TaRAP2.1Lmut.

### Example 4—Molecular Models of the AP2 Domain of TaRAP2.1L and Recognition Selectivity of the DRE, CRT and GCC-Box Cis-Elements

To explain determinants of the unusual binding selectivity of TaRAP2.1L models of TaRAP2.1L were generated in complexes with three different cis-elements. Modelling of the TaRAP2.1L interacting with DNA was performed using the AP2 domain of AtERF1 (1gcc:A). The positional sequence identity and similarity values between the two proteins were 35% and 55%, respectively, indicating that modelling was reliable. Analyses using PROCHECK and Prosa2003 indicated that the stereochemistry of protein structural models was satisfactory and that the 3D models were of a high quality.

Molecular models revealed that TaRAP2.1L contained one α-helix and three anti-parallel β-sheets that folded into a global scaffold of the ‘alpha and beta protein’ class. Models of the AP2 domains were generated in the presence of cis-elements and allowed us to envisage how individual DNA molecules (DRE) were bound and which protein determinants underlined cis-element recognition selectivity. The modelling revealed that anti-coding strands of DNA molecules bound through a series of highly conserved residues exposed on one side of the three anti-parallel β-sheets, whereby in all instances Gly, Arg and Trp residues mediated contacts between cis-elements and AP2 domains. Arg55 was always involved in binding of the first base (either A or G) of the coding strands of cis-elements, forming separations between 2.8 Å and 3.5 Å. A second residue, Gly51, always participated in binding of the anti-codon strand by interacting with a diphospho-group of the backbone inter-connecting the two last bases (T-G or G-G). This binding formed tight separations at 2.6 Å. The same binding characteristics were found with AtERF1 in complex with the GCC-box). The Arg55 and Gly51 residues participated in binding of bases that differed between the three elements, and thus were responsible for relaxed DNA-binding specificity of TaRAP2.1L. Another residue that interacted with all three elements was Arg72, although this was always with a base (G) of a coding-strand that was invariant between three cis-elements. Two other residues, Arg53 and Arg65 either participated in binding (DRE) or partially participated (CRT), while in the case of the GCC-box, neither additional Arg was involved, top-right panel). Other residue that mediated contacts in binding of CRT was Trp74, meaning that this element should be bound tightly, as indicated by experimental EMSA. Conversely, Trp74 did not seem to be involved in binding of DRE and GCC-box cis-elements. It was concluded that the tighter binding of DRE and CRT elements by TaRAP2.1L is related to participation of at least four to five residues (Gly51, Arg53, Arg55, Arg72 and Trp74), while only three residues (Gly51, Arg55 and Arg72) participated in binding of the GCC-box.

The TaRAP2.1L gene was isolated from leaf and spike tissues of wheat subjected to drought and heat. It was found to be a component of the ABA-mediated response to abiotic stresses. TaRAP2.1L gene expression was induced by ABA and abiotic stresses and, in the absence of stress it was strongly expressed in desiccating grain, a tissue with elevated levels of ABA. The TaRAP2.1L gene showed a relatively high basal level of expression in all tested tissues. These findings, and the fact that TaRAP2.1L was induced by stress quicker than the DREB/CBF activator TaDREB3, were inconsistent with the proposed ‘capping’ role for RAP2.1 in Arabidopsis.

Proteins comprising RAP2.1 and DEAR-like groups are smaller than most of DREB/CBF activators. Each of them contains a single AP2 DNA-binding domain, a conserved motif of unknown function in the middle of the protein sequence and a C-terminal EAR motif. No obvious activation domain or motifs, often characterised by alternating or enriched acidic and hydrophobic residues, were found in TaRAP2.1L, although it has a short stretch of acidic residues at the C-terminal end of the protein, downstream from the EAR motif. However, in a yeast activation assay neither WT TaRAP2.1L nor its mutated form behaved as activators.

Different selectivity of cis-element recognition by positive and negative DREB/CBF regulators is also inconsistent with the proposed capping role of this protein. A capping function implies that DREB/CBF activators and DREB/CBF repressor(s) share the same DNA binding specificity to control expression levels of the same target genes. However, in contrast to most studied wheat DREB/CBF activators, TaRAP2.1L can interact with a GCC box and thus can potentially regulate a larger number of genes than the DREB/CBF activators, including plant defense genes regulated by ERFs. It has previously been reported that some DREB/ERFs containing an EAR motif can bind to both a GCC-box and DRE/CRT elements. For example, a soybean (Glycine max L.) GmERF4 protein, containing the EAR motif was able to recognise both a GCC-box and DRE/CRT elements in vitro (Zhang et al., 2010, Mol Biol Rep 37, 809-818).

Structural models of the TaRAP2.1L AP2 domain showed that a mutual interplay of residues within the secondary structure elements of the domain that form three antiparallel β-sheets, could influence binding of three distinct, yet related DRE, CRT, and GCC-box cis-elements. Variant bases of anti-codon strands are bound by a highly conserved Arg55 residue, while invariant bases are bound by other conserved Arg residues that not always participate in binding. Structural comparisons of molecular models of the AP2 domain of TaRAP2.1L in complex with three DNA cis-elements highlighted that the flexibility of residues that line the anti-parallel β-sheets, and the mutual interplay of residues within these secondary elements could influence the strength of binding of the different cis-elements. These hypotheses could be tested by generating variant molecules of the TaRAP2.1L AP2 domain through site-directed mutagenesis and plant transformation. Variant forms could be designed to attempt to either alter cis-element binding selectivity or abolish cis-element binding altogether. It may be expected that folding patterns of the mutated TaRAP2.1L protein could be different from the one of the wild-type protein; this may cause variations in protein-protein interactions and thus regulatory properties of this modified TF. To predict mechanistic approaches to recognition of cis-elements, MD simulations of the key residues participating in binding of cis-elements by TaRAP2.1L were performed. The models generated in complex with cis-elements predicted that interaction of TaRAP2.1L with a GCC-box is possible but weaker than when binding to DRE and CRT-elements. This agreed with the experimental observation that yeast containing GCC-box integrated-to-genomic DNA was growing slower than yeast containing the DRE and CRT-elements, when transformed with a TaRAP2.1L expression construct. Weaker interaction with the GCC-box was also detected by EMSA.

### Example 5—Constitutive and Stress-Responsive Up-Regulation of TaRAP2.1L Negatively Influence Barley as Well as Wheat Growth and Frost Tolerance

To investigate the influence of TaRAP2.1L on wheat development and performance under different stress conditions, the TaRAP2.1L gene was overexpressed in barley and wheat under the control of constitutive and stress-inducible promoters, respectively. Eleven independent barley lines were generated using a pUbi-TaRAP2.1L construct, with most lines estimated to contain 2 to 6 copies of the transgene. Expression levels of the transgene were examined by RNA-blot analysis using total RNA from leaves. It is of note that transgene expression levels usually vary in independent lines due to positional effects of insertion, damage of constructs during transformation, etc. T0 barley lines with strong transgene expression produced few or no seeds and could not be used in further analyses. Most of the T1 barley plants grew significantly slower than control WT plants, had dwarf phenotypes and up to 6-week delayed flowering.

The seed of three T0 barley lines with mild levels of transgene expression in leaves were used for analysis of frost tolerance at a seedling stage. Genomic PCR using transgene-specific primers and northern blot hybridization were used for each plant to confirm transgene presence and expression, respectively. Null-segregants were removed from the analyses. Two from three of the tested lines had decreased frost tolerance, while the third line showed a similar frost survival rate as the control WT plants. The likely explanation for a weak decrease of frost tolerance in barley is that the selection for seed-producing lines yielded lines with low transgene expression levels and therefore weak phenotypes. Because a significant negative influence of the TaRAP2.1L transgene was observed under well-watered conditions, drought tolerance experiments were not performed for these lines.

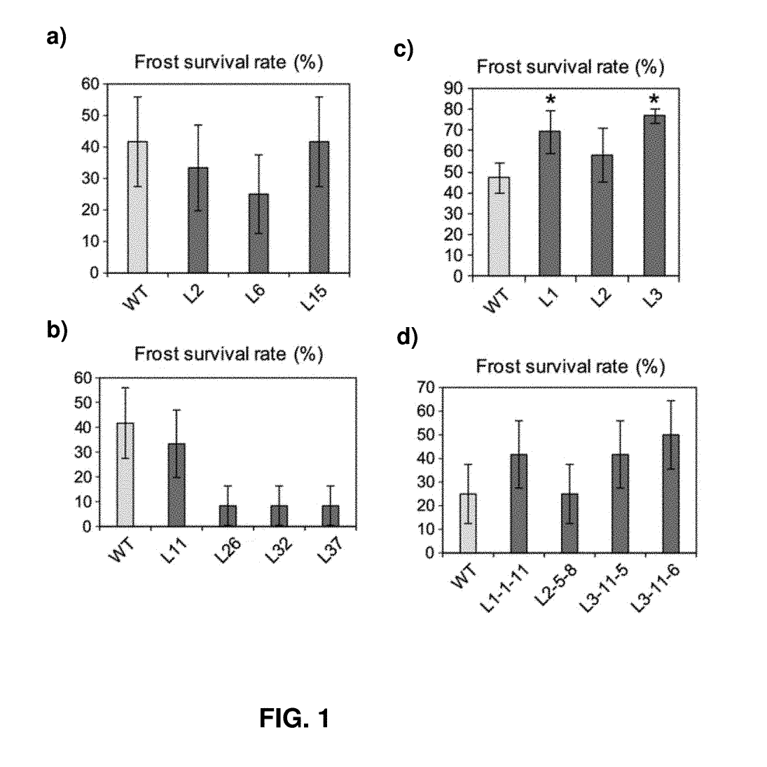

wheat plants containing pUbi-TaRAP2.1L construct were also generated, however very few T0 plant had survived, and those which survived produced small amounts of seeds that cannot be further analysed. With the aim to decrease differences in the developmental phenotypes of control and wheat plants, a stress inducible promoter of barley, Dehydrin 8 (Dhn8) was also used for over-expression of TaRAP2.1L. The structure and properties of the Dnh8 promoter are similar to those of the TdCor410b promoter, showing strong induction by cold, drought and salinity, a moderate level of constitutive expression in leaves and a high level of expression in the developing grain. A total of 12 independent lines of wheat (cv. Bobwhite) were generated. Analysis of T1 progeny demonstrated a mild negative influence of the transgene on plant development, whereby plants produced less seeds than the control WT plants. The frost tolerance of the T1 progeny of four independent wheat lines and control WT plants was compared in a frost survival test. All four tested lines showed lower survival rates than the control WT plants (FIG. 1A).

### Example 6—the EAR Domain of TaRAP2.1L is Responsible for a Negative Effect on Growth and Stress Tolerance of Wheat Lines

The molecular variant TaRAP2.1Lmut was shown to have the same DNA binding specificity as WT TaRAP2.1L, and therefore could potentially compete with a WT protein for binding to stress-responsive promoters. For these reasons, TaRAP2.1Lmut was overexpressed in wheat under a strong constitutive pUbi promoter.

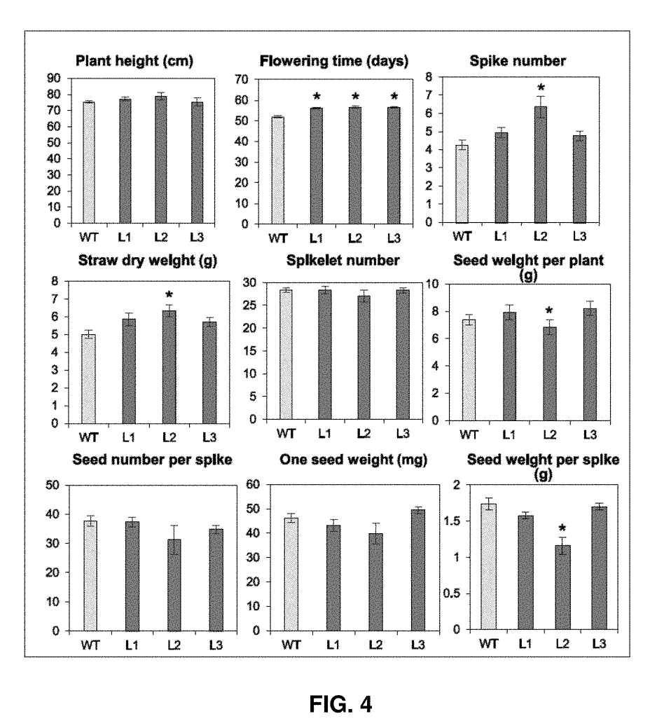

Independent pUbi lines (a total of 17) were generated in the elite wheat cultivar Gladius. Single copy lines were identified by genomic Q-PCR, as outlined in Supporting experimental procedures, and the lines with strong constitutive expression of the transgene were analysed using northern blot hybridization and Q-PCR. Comparisons of growth and yield parameters of the WT plants and T1 progeny of selected wheat lines of the same genetic background under well-watered conditions, revealed no significant negative influence of the transgene on plant growth, a mild delay of flowering (about 3 days), and no significant yield penalty in two of three tested independent lines (FIG. 1A; FIG. 4). However, two of the three tested lines demonstrated a significant improvement in frost tolerance compared to the control WT plants (FIG. 1B). The frost tolerance experiment was repeated three times using 12 T1 plants for each line in each of three experiments. Only results obtained for plants with confirmed presence and expression of the transgene (24-30 plants per line) were used (FIG. 1C). The frost tolerance test was repeated using four T3 homozygous sub-lines, 12 plants per sub-line. The results of this experiment correlated well with the data from frost tests of the T1 generation plants (FIG. 1D).

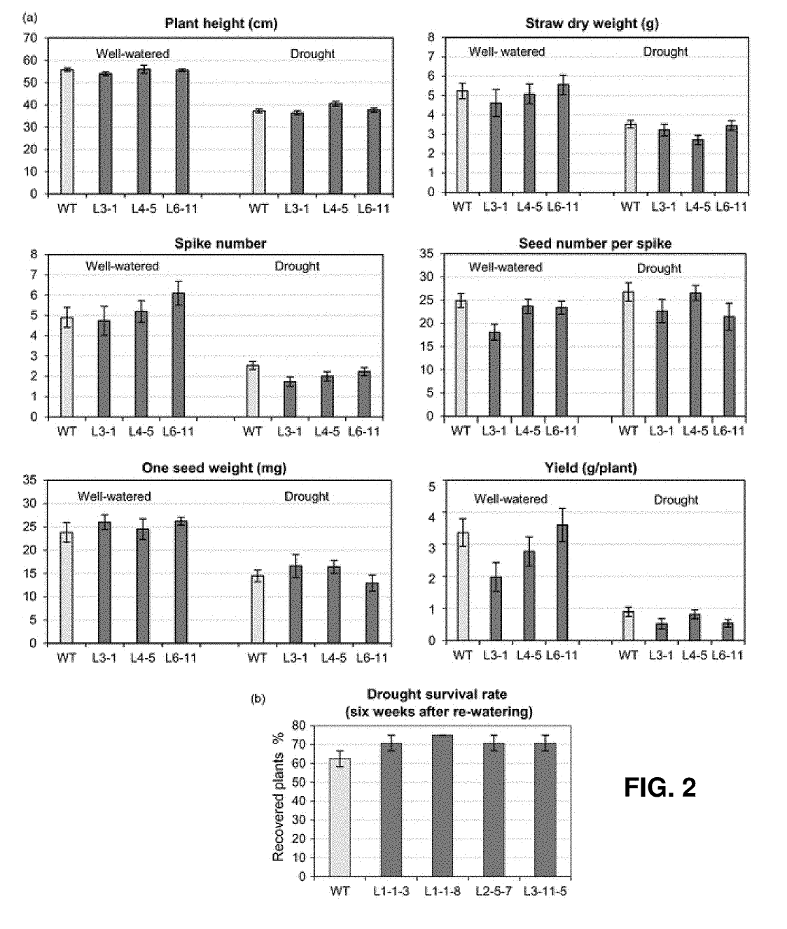

Phenotyping of the T2 progeny of the pUbi-TaRAP2.1Lmut lines under well-watered and drought conditions was performed in two large containers, using 10-16 plants per line in each container. Evaluation of grain yield using large containers was performed under two different water regimes. No significant differences in plant growth and yield components were found between control and plants under well-watered conditions (FIG. 2A). Under the drought conditions, the lines were 5-15% taller than the control WT plants but produced less tillers. As a result, the yields of the control WT and plants were not significantly different. In other experiment, the survival rates of seedlings subjected to severe drought conditions (water not available for 10 days) at six weeks after re-watering, performed by 5 to 15% better than those of the control WT plants (FIG. 2B), indicating that overexpression of TaRAP2.1Lmut improved the ability of wheat plants to recover from drought stress. The difference in appearance of control and plants was larger at two weeks than at six weeks after re-watering, reflecting a difference in speed rather than in rate of recovery from a drought event between and control plants. Taking in consideration that drought tolerance of an already extremely drought tolerant cultivar was attempted, these data suggested that if there was a need to increase drought tolerance of drought sensitive wheat without negative influence on the phenotype, this was plausible.

Both strong constitutive and stress-inducible activation of TaRAP2.1L in barley and wheat plants had a negative influence on plant growth and time to flowering, and significantly decreased plant frost tolerance. A protein competitor of TaRAP2.1L was used to down-regulate endogenous TaRAP2.1L gene expression, instead of a gene silencing approach. In cases where the generation of mutants or gene deactivation on an RNA level is difficult to achieve, the use of protein competitors with altered functionality can provide an efficient alternative approach for reducing, switching off or even reversing gene function. The protein competitor TaRAP2.1Lmut used in this work has a functional DNA-binding domain and a deactivated EAR motif. It was expected that this protein variant would be either itself converted from repressor to activator, or would play a role of a ‘passive activator’ by competing with WT TaRAP2.1L for binding to promoters of target genes, thus decreasing or preventing their repression. As a first step, the abilities of TaRAP2.1L and TaRAP2.1Lmut to bind different cis-elements were compared. TaRAP2.1Lmut demonstrated the same DNA binding properties as TaRAP2.1 in an Y1H assay and EMSA, suggesting no influence of the mutated EAR motif on the strength or specificity of DNA binding.

### Example 7: Over-Expression of TaRAP2.1Lmut Enhances Expression of Stress-Responsive Genes in Wheat Leaves

Experimental Analysis of Plants:

wheat and barley plants were grown in either a growth room (cold and drought tests) or a glasshouse (characterisation of plant phenotypes). Growth room temperatures were maintained at 24° C. during the 12-h day and 18° C. during the night, and the average relative humidity was 50% during the day and 80% during the night. WT plants were used as controls. Seeds were germinated on moist paper in Petri dishes at room temperature for 3 days and transferred to containers with soil. For phenotyping under well-watered conditions, the plants were grown either in small pots (8×8×10 cm), one plant per pot (T1 generation) or in large containers (T2 generation). The size of each container was 120×80×40 cm and the distance between plants was 8 cm. Each container had 10 sub-plots, flanked by a border row (WT plants) on each short side of the container. These border plants were not used in the experiment. Containers were equipped with an automatic watering system and four soil water tensiometers (gypsum blocks) were installed at 0.1 and 0.3 m soil depths, and connected to a data logger for continuous monitoring of soil water tension. Plant height, number of tillers and spikes, plant biomass, seed number and seed weight were recorded for each plant. In segregating heterozygous lines (T1 and some T2), nulls were selected based on the results of PCR analyses for transgene presence and/or transgene expression estimated by northern blot hybridisation. Null segregants were excluded from the data analysis.

Plants were grown in rows, eight plants per row for each line and WT with three or four randomised blocks in each container comprising in total 16 biological replicates for each line and WT plants. Experimental plants were flanked by a border row of WT plants on each short side of the container. The experimental design was identical for each container. No significant differences were found in plant growth between the three or four blocks in several preliminary experiments, and therefore all replicates for each line and WT plants were used, to calculate confidence intervals and means for individual measurements.

For cold treatment, control and seedlings were grown for three weeks in pots of soil in a growth room, transferred to a cold cabinet and treated according to freezing tolerance test programs for wheat and barley (Kovalchuk et al., 2013, Plant Biotechnol J 11, 659-670). For frost survival tests, three-week old seedlings were exposed to the gradual temperature decreases to a minimum of −6° C. and then slowly returned to a maximum of 18° C. Leaf tissue was collected before stress (in the case of both constitutive and inducible promoters) and after the temperature had decreased to 4° C. (for inducible promoter only). When the temperature reached −5° C., plants were sprayed with a 2 g/L solution of Snomax (York Snow, Victor, N.Y.) to initiate simultaneous ice crystallisation before the temperature reached −6° C. The temperature was afterwards decreased to −6° C. for 10 h or 3 h, for barley and wheat seedlings, respectively. When the temperature returned to 18° C., plants were returned to the growth room to recover. The number of surviving plants was recorded after two weeks of recovery. Surviving plants were re-potted and transferred to a glasshouse for seed production.

For drought survival tests, one control plant and one plant for each line were grown in the same pot; 10-12 pots were used in each experiment, and each experiment was repeated three times for T1 lines. Watering was withheld after three weeks of plant growth at well-watered conditions and plants were re-watered after 10-14 days, when they showed symptoms of severe stress (strong wilting, leaf rolling and severe dehydration). The number of surviving plants was recorded after two and six weeks of recovery.

Assessment of plant yield under mild drought was performed in large containers as described above. Watering was withheld at the beginning of booting. During flowering, plants showed mild symptoms of stress; soil water tension at this point was between −0.3 and −0.4 MPa. At the 10th day after the end of flowering, plants were re-watered and mild watering continued until harvest. Plant height, number of tillers and spikes, plant biomass, seed number and seed weight were recorded for each plant. WT plants were used as a negative control

Results and Discussion:

Six of eight tested stress-responsive genes (e.g. TaCor14B, TaRAB17 and TaCor80), which are known to be directly or indirectly regulated by DREB TFs, were found to have significantly higher levels of expression in the leaves of unstressed wheat plants compared to the control WT plants. The levels of expression of these genes reflected closely the levels of transgene expression, implying a direct regulatory effect of TaRAP2.1Lmut on their promoters. These data suggested that the physiological mechanism, which improved drought/desiccation tolerance of lines transformed with the TaRAP2.1Lmut protein, may be similar to that reported in the literature for most of other DREB activators. In addition TaCor410b and WLT10 genes showed no regulation by the transgene, and the transcript levels of endogenous TaRAP2.1L were elevated.

TaRAP2.1Lmut was constitutively overexpressed in the elite Australian wheat cultivar Gladius and phenotypes of resulting lines were evaluated for frost and drought tolerance at a seedling stage, and for yield under well-watered conditions and moderate drought. In contrast to TaRAP2.1L, constitutive overexpression of TaRAP2.1Lmut had no significant influence on plant development under well-watered conditions. This happened, presumably, because binding of the mutated form of TaRAP2.1L to cis-elements of stress-responsive promoters did not influence plant development in the absence of stress. However, several changes were observed under drought conditions. plants grew taller, and had a slightly increased single grain weight. A reduction in the number of tillers likely reduced potential yield improvement. Increased plant height under drought and no influence of TaRAP2.1Lmut overexpression on wheat growth under well-watered conditions suggests a possible involvement of the EAR domain in plant growth suppression.

Although lines demonstrated no yield gains compared to those of WT wheat, clear increases in tolerance to severe stresses was achieved. Frost survival rates of wheat were significantly better than frost survival rates of control plants and these results were reproducible across two generations. A small improvement in terminal drought survival rate was also observed. It was not easy to detect differences in drought tolerance of and control wheat plants because of the genetic background cultivar Gladius, used in these experiments, is already a drought-tolerant cultivar. Nevertheless, plants recovered more quickly than control WT plants after re-watering and had slightly higher survival rates.

Enhanced stress tolerance of wheat resulted from significant up-regulation of several known target genes of DREB/CBF activators. It is of note that the TaRAP2.1L endogene was also up-regulated in plants, suggesting a feed-back mechanism of the TaRAP2.1L promoter regulation by its own protein product. This is in a good agreement with the reported data obtained by chromatin immuno-precipitation and transient expression assays on the ability of RAP2.1 to bind and repress its own promoter (Dong and Liu, 2010). Any potential downstream effects of the elevated expression levels of endogenous TaRAP2.1L in wheat plants were neutralised by the more abundant protein variant, resulting in an overall up-regulation of target genes and stress tolerance improvement.

Constitutive overexpression of TaRAP2.1Lmut, in contrast to constitutive overexpression of most DREB/CBF activators, did not influence plant development and yield but increased the stress tolerance of wheat. This presumably resulted from activation of multiple and diverse sets of stress-responsive genes. It remains, however, unclear if TaRAP2.1Lmut works as an activator itself or functions through prevention of binding of native TaRAP2.1L to promoters of stress-responsive target genes

The disclosure has been described with reference to various specific and preferred embodiments and techniques. However, it should be understood that many variations and modifications may be made while remaining within the spirit and scope of the disclosure.

What is claimed is:

1. A method of improving abiotic stress tolerance of a crop plant, the method comprising increasing the expression of a polynucleotide encoding a monocot Rap2.1L polypeptide, wherein the Rap2.1L polypeptide does not comprise a functional EAR motif.

2. The method of claim 1, wherein the crop plant is tolerant to drought, frost or cold stress.

3. The method of claim 1, wherein the crop plant is selected from the group consisting of wheat, barley, rice, millet, maize, and sorghum.

4. The method of claim 1, wherein the Rap2.1L polynucleotide is selected from the group consisting of (i) a polynucleotide encoding an amino acid sequence comprising SEQ ID NOS: 1, 3, 5, 7, 9, 11, 13, 15 and 17 or an amino acid sequence that is at least 95% identical to one of SEQ ID NOS: 1, 3, 5, 7, 9, 11, 13, 15 and 17 (ii) a polynucleotide that hybridizes under stringent hybridization conditions to a polynucleotide comprising SEQ ID NOS: 2, 4, 6, 8, 10, 12, 14, 16 and 18 (iii) a polynucleotide that encodes an amino acid sequence that is at least 90% identical to SEQ ID NOS: 1, 3, 5, 7, 9, 11, 13, 15 and 17 and not comprising a functional EAR motif, (iv) a polynucleotide encoding a polypeptide comprising one or more deletions or insertions or substitutions of amino acids in the EAR motif present in a polypeptide sequence selected from the group consisting of SEQ ID NOS: 1, 3, 5, 7, 9, 11, 13, 15 and 17.

5. The method of claim 4, wherein the expression of the polynucleotide encoding a polypeptide having at least 95% identity to SEQ ID NO: 1 is increased by transforming the plant with a recombinant polynucleotide operably linked to a heterologous promoter.

6. The method of claim 4, wherein the expression of an endogenous polynucleotide encoding a polypeptide having at least 95% identity to SEQ ID NOS: 1, 7, 9, 11, 13, 15, or 17 is increased by upregulating a regulatory element operably associated with the endogenous polynucleotide.

7. The method of claim 4, wherein the expression of the polynucleotide is increased by expressing the polynucleotide under a tissue preferred regulatory element.

8. The method of claim 1, wherein the abiotic stress tolerance comprises a characteristic selected from the group consisting of wilting avoidance, improved photosynthetic performance, increased chlorophyll content, increased photosynthetic rate, improved stomatal conductance, carboxylation efficiency, an increase in grain size, an increase in grain weight, an increase in grain yield, an increase in grain filling rate, and an increase in biomass.

9. The method of claim 1, wherein the abiotic stress tolerance is an increase in drought tolerance during vegetative and/or reproductive stages.

10. The method of claim 1, wherein the grain weight is increased in relation to a control plant not having an increased expression of the polynucleotide.

11. The method of claim 1, wherein the plant is a monocot.

12. The method of claim 1, wherein the plant is selected from the group consisting of maize, rice, wheat, barley, rye, millet, and sorghum.

13. The method of claim 1, wherein the plant is a dicot.

14. The method of claim 1, wherein the plant is soybean or brassica.

15. A method of improving yield of a plant, the method comprising increasing the expression of a polynucleotide that encodes a polypeptide comprising the amino acid sequence selected from the group consisting of SEQ ID NOS: 1, 7, 9, 11, 13, 15, or 17 or an allelic variant thereof.

16. (canceled)

17. (canceled)

18. (canceled)

19. A recombinant expression cassette, comprising the polynucleotide of claim 1, wherein the polynucleotide is operably linked to a regulatory element, wherein the expression cassette is functional in a plant cell.

20. A host cell comprising the expression cassette of claim 19.

21. A plant comprising the recombinant expression cassette of claim 19.

22. A plant comprising a plant regulatory element that operably regulates the expression of a polynucleotide encoding a polypeptide comprising the amino acid sequence of SEQ ID NOS: 1, 7, 9, 11, 13, 15, or 17 or a variant or an ortholog thereof, wherein the regulatory element is heterologous to the polynucleotide and wherein the plant exhibits substantially normal phenotype as compared to a control plant.

23. The plant of claim 21, wherein the plant is a wheat, rice, barley, maize, rye, millet, sorghum, triticale, oat, teff, wild rice, spelt, buckwheat, turf grass, rye grass, switchgrass, Miscanthus, or Festuca plant.

24. (canceled)

25. (canceled)

26. (canceled)

27. (canceled)
